(19) United States
(12) Patent Application Publication
(10) Pub. No.: US 2026/0250639 A1
(43) Pub. Date: Aug. 27, 2026

(54) REPROGRAMMING METHOD

(71) Applicant: Babraham Institute, Cambridge (GB)

(72) Inventors: Maria A. CHRISTOPHOROU, Cambridge (GB); Johanna GRINAT, Cambridge (GB); Robert WALMSLEY, Cambridge (GB)

(21) Appl. No.: 19/183,196

(22) Filed: Apr. 18, 2025

Related U.S. Application Data
(63) Continuation of application No. PCT/GB2023/052750, filed on Oct. 20, 2023.

(30) Foreign Application Priority Data
Oct. 21, 2022 (GB) 2215629.3
Jun. 28, 2023 (GB) 2309737.1

(51) Int. Cl. C12N 5/074; A61K 38/17

(52) U.S. Cl. CPC C12N 5/0696; A61K 38/1709; C12N 2501/065; C12N 2501/603; C12N 2501/605; C12N 2506/08

(57) ABSTRACT

The invention relates to methods of non-cell autonomous modulation of reprogramming comprising providing a non-cell autonomous reprogramming factor which derives from a cell other than the somatic cell to be reprogrammed. The invention further relates to the non-cell autonomous reprogramming factor, and analogues thereof, which is derived from the cell other than the somatic cell to be reprogrammed, and its use in methods of reprogramming a somatic cell in vitro. Also provided is the non-cell autonomous reprogramming factor, or analogues thereof, for use in cell and tissue treatment, rejuvenation, regeneration and repair.

This application is a bypass continuation of International Application No. PCT/GB2023/052750, filed Oct. 20, 2023, which claims the benefit of priority of United Kingdom Application No. 2309737.1, filed Jun. 28, 2023, and United Kingdom Application No. 2215629.3, filed Oct. 21, 2022, the contents of which are incorporated by reference as if written herein in their entireties.

### FIELD OF THE INVENTION

The invention relates to methods of non-cell autonomous modulation of reprogramming comprising providing a non-cell autonomous reprogramming factor which derives from a cell other than the somatic cell to be reprogrammed. The invention further relates to the non-cell autonomous reprogramming factor, and analogues thereof, which is derived from the cell other than the somatic cell to be reprogrammed, and its use in methods of reprogramming a somatic cell in vitro. Also provided is the non-cell autonomous reprogramming factor, or analogues thereof, for use in cell and tissue treatment, rejuvenation, regeneration and repair.

### BACKGROUND OF THE INVENTION

The discovery that somatic cells can be reprogrammed to a pluripotent state and instructed to differentiate into variety of cell types promises to revolutionise regenerative medicine by rendering it possible to repair or replace diseased and damaged tissues. Reprogramming can be achieved via transduction of the “Yamanaka” transcription factors Oct4, Sox2, Klf4 and c-Myc, or combinations of similar factors, into the somatic cells. Using this method, a relatively small proportion of somatic cells reprogramme successfully to become induced pluripotent stem cells (iPSCs) and, while the cell-intrinsic (i.e. cell autonomous) mechanisms that mediate reprogramming have been studied extensively, we still lack rational approaches to enhance reprogramming efficiency. The majority of the cells, which do not emerge as iPSCs, are deemed to have failed reprogramming and are typically ignored.

The transduction of Yamanaka factors into somatic cells to be reprogrammed leads to the genetic manipulation of said cells, and can predispose them to cancerous transformation and teratoma formation. The Yamanaka factor, c-Myc in particular has been implicated in this process. Furthermore, the transduction of Yamanaka factors in vivo to bring about iPSC reprogramming in tissue repair, regeneration and rejuvenation can be challenging.

There is therefore a great need to identify non-cell autonomous mechanisms and processes which avoid, or reduce the requirement for, the genetic manipulation of somatic cells in methods of reprogramming. Identifying the factors involved in these non-cell autonomous reprogramming mechanisms/processes (i.e. non-cell autonomous reprogramming factors) may allow their isolation or synthesis and administration to biological systems with the ultimate goal of achieving highly efficient reprogramming while minimising genetic manipulation of somatic cells by transduction of reprogramming factors (such as some of the Yamanaka factors), thus minimising the risk of cancerous transformation and teratoma formation. The modulation of these non-cell autonomous reprogramming mechanisms and use of the factors which modulate them may then be applied towards the achievement of efficient and safe reprogramming technologies in vitro and in vivo, impacting the areas of iPSC reprogramming and tissue repair, regeneration and rejuvenation.

### SUMMARY OF THE INVENTION

According to a first aspect of the invention, there is provided a method of non-cell autonomous modulation of reprogramming, comprising providing a non-cell autonomous reprogramming factor and a somatic cell to be reprogrammed, wherein said non-cell autonomous reprogramming factor derives from a cell other than the somatic cell to be reprogrammed.

In one embodiment, the non-cell autonomous reprogramming factor derives from a non-reprogramming cell, such as a bystander cell. Thus in a further embodiment, the non-cell autonomous reprogramming factor does not derive from the somatic cell to be reprogrammed, such as does not derive from the reprogramming cell. In a yet further embodiment, the non-cell autonomous reprogramming factor is released from the cell other than the somatic cell to be reprogrammed, such as is released from a non-reprogramming cell and/or a bystander cell. In another embodiment, the non-cell autonomous reprogramming factor is an analogue of the non-cell autonomous reprogramming factor derived from non-reprogramming cells, such as an isolated or synthetic (e.g. in vitro synthesised) functional analogue.

According to a further aspect of the invention, there is provided the non-cell autonomous reprogramming factor defined herein, or an analogue thereof, for use in a method of treating and/or ameliorating a degenerative disease or disorder or a method of rejuvenating, repairing or regenerating a tissue or organ, wherein said method comprises reprogramming a somatic cell in vivo.

In another aspect, there is provided the non-cell autonomous reprogramming factor defined herein, or an analogue thereof, for use in a method of treating and/or ameliorating a degenerative disease or disorder or a method of rejuvenating, regenerating or repairing a tissue or organ, wherein said non-cell autonomous reprogramming factor derives from a cell other than the somatic cell to be reprogrammed in vivo.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: Schematic of method of reprogramming of mouse neural stem cells into induced pluripotent stem (iPS) cells. The mouse neural stem cell (NSC) line NSO4G harbours the GFP transgene under the control of Oct4 regulatory elements. NSCs do not express Oct4 and are therefore GFP-negative. Upon reprogramming, iPSCs grow in colonies and express GFP, allowing their visualisation and isolation from the remaining, non-reprogrammed cells.

FIG. 2: Temporal analysis of the expression and activation of PADI4 during the reprogramming process. A) Real time quantitative PCR (RT-qPCR) for the levels of Padi4 and Nanog during the reprogramming process. NSCs do not express Padi4 or Nanog prior to reprogramming. Padi4 expression is induced after transduction of the Yamanaka factors and precedes the expression of Nanog. Expression relative the housekeeping gene Ubiquitin C (UbC). B) Immunoblot analysis for PADI4, H3Cit and GFP from the pre-iPS stage (day 6) until the end of reprogramming (day 15). H3Cit is used as a measure of PADI4 activation (catalytic activity) and GFP is used as a reporter for the expression of endogenous mouse Oct4 protein (which is distinct from the exogenous Oct4 provided as a Yamanaka factor). PADI4 protein is expressed at very low levels in pre-iPS cells, but it is stabilised and activated (see H3Cit) after addition of 2i. Expression and activation of PADI4 precede the expression of Oct4 (GFP). C) Schematic representing the data in FIGS. 1B and 1C collectively.

FIG. 3: Pharmacological or genetic inhibition of PADI4 reduces reprogramming. A-C) Yamanaka factor-mediated reprogramming of NSO4G neural stem cells. D) Yamanaka factor-mediated reprogramming of human fibroblasts. A) Schematic representation of the experimental design for data in panels B and C. B) Flow cytometry plots (left) and quantification data (right) for the number of GFP-positive cells at the end of reprogramming, in the presence of PADI4 or control shRNAs. C) Flow cytometry plots (left) and quantification data (right) for the number of GFP-positive cells at the end of reprogramming, in the presence of CI-amidine or vehicle control. D) Quantification of iPS colonies, as a measure of the degree of reprogramming, after Yamanaka factor-mediated reprogramming of human fibroblasts, in the presence of two independent PADI4 inhibitors (CI-amidine or GSK484) or vehicle controls. The inhibitors were administered throughout the course of reprogramming.

FIG. 4: PADI4 expression and activity are found in the non-reprogramming cells of reprogramming cultures and surround the iPS colonies. A-C) Yamanaka factor-mediated reprogramming of NSO4G neural stem cells. D) Yamanaka factor-mediated reprogramming of human fibroblasts. A) Immunocytochemistry analysis of reprogramming NSC cultures, over the time course of reprogramming from the pre-iPS stage (day 6) until the end of reprogramming. Citrullinated histone H3 (H3Cit) shown in red. E-cadherin, marking the reprogramming cells and iPS colonies shown in green. DAPI stain for DNA shown in blue. B) Immunoblot analysis of pre-iPS cells (right panel, lane 1) and iPS cells at the end of reprogramming as a total population (right panel, lane 2) or sorted by flow cytometry (left panel) on the basis of GFP expression (right panel, lane 3: GFP-positive, lane 4: GFP negative). PADI4 expression and activity (H3Cit) are in the GFP-negative, non-reprogrammed cells. C) Immunocytochemistry analysis of NSO4G reprogramming cultures at the time course mid-point (day 12) for the pluripotency marker Nanog (green) and H3Cit (red), showing that the H3Cit-positive cells are mutually exclusive with Nanog-positive iPS cells. DAPI stain for DNA shown in blue. D) Immunocytochemistry analysis of human fibroblast reprogramming cultures over the course of the reprogramming time course, from the pre-iPS stage (day 7) until consolidation of reprogramming (day 20). E-cadherin staining (green) marks the iPS colonies. H3Cit staining shown in red and DAPI stain for DNA shown in blue.

FIG. 5: Medium conditioned by reprogramming cultures increases reprogramming of recipient cells. A) Schematic of experimental design. Reprogramming cultures received medium that had been conditioned by other reprogramming cultures for 24 h (bottom panel) or control medium conditioned in an empty dish under identical conditions for 24 h (top panel). The degree of reprogramming within the recipient cultured was assessed by flow cytometry-based quantification of GFP-positive cells at the end of the reprogramming protocol. B) Flow cytometry-based quantification of GFP-positive iPS cells after culturing reprogramming cells as described in (A).

FIG. 6: Citrullinated chromatin is extracellular. A) Immunocytochemistry analysis of reprogramming cultures at the mid-point of NSC reprogramming (day 12). A single iPS colony is shown. GFP (green) marks the iPS cells. H3Cit shown in red and DAPI stain for DNA shown in blue. B) Enlarged area of the periphery of the iPS colony shows unusual nuclear morphology of the H3Cit-positive cells. Immunocytochemistry analysis, H3Cit shown in red. C) Immunocytochemistry analysis using conditions optimised for the visualisation of extracellular, NET-like chromatin shows extracellular chromatin fibres containing H3Cit (red) and DNA (DAPI, blue). NET-like chromatin is highly decondensed, resulting in faint DAPI stain. High exposure of the DNA stain is shown to visualise the extracellular DNA fibres.

FIG. 7: Citrullinated histones are found in culture medium conditioned by reprogramming cultures. Immunoblot analysis for total histone H3 (top) and two different citrullination marks on histone H3 (middle and bottom) on conditioned medium of NSC reprogramming cells cultured in the presence or absence or the PADI4 inhibitor CI-amidine. Data from two independent experiments are shown. Citrullinated histone H3 is readily detected in the conditioned medium and inhibited by CI-amidine.

FIG. 8: NET-like citrullinated chromatin is induced during in vivo reprogramming and associates with tissue reprogramming. A) Immunohistochemical analysis of pancreas tissue from the i4F in vivo reprogramming mouse (Abad et al. (2013) Nature) after induction of Yamanaka factor expression within the tissue. Oct4 staining (red) shows areas of reprogramming within the tissue. H3Cit-positive cells shown in green. B) Enlarged view of the H3Cit-positive cells (white square within panel (A)) shows NET-like structures within the reprogramming areas of the tissue. H3Cit shown in green, DAPI stain for DNA shown in blue. High exposure of the DAPI stain presented to allow visualisation of decondensed NET-like chromatin.

FIG. 9: Histone citrullination and NET-like chromatin release are induced during the regenerative phase in a model of mouse digit tip amputation and regeneration. A) Immunohistochemical analysis of uninjured mouse digit tip tissue and at different time points post amputation (days post amputation, DPA). H3Cit shown in green. DAPI stain for DNA shown in blue. B) Immunohistochemical analysis of mouse digit tip tissue at 7 days post amputation. H3Cit shown in green. DAPI stain for DNA shown in white. Yellow arrowheads show areas of NET-like citrullinated and decondensed chromatin structures.

FIG. 10: Inhibition of extracellular chromatin component-sensing pathways inhibits reprogramming. A) Schematic representation of the extra-nuclear DNA sensing pathway cGAS/STING (left panel). Flow cytometry-based quantification of GFP-positive cells at the end of NSC reprogramming (day 15), in the presence of STING inhibitor H-151, vehicle control (DMSO) or no treatment (right panel). B) Schematic representation of the signalling pathway downstream of the Damage Associated Molecular Pattern (DAMP)-sensing Toll-Like Receptor 2 (TLR2; left panel). Flow cytometry-based quantification of GFP-positive cells at the end of NSC reprogramming (day 15), in the presence of TLR2 inhibitor MMG-11, vehicle control (DMSO) or no treatment (right panel). C) Colony count data for quantification of reprogramming of human fibroblast cells at the end of reprogramming (day 40) after treatment with STING inhibitor H-151 (left panel), TLR2 inhibitor MMG-11 (right panel) or vehicle control (DMSO).

FIG. 11: Degradation of extracellular DNA in the medium of reprogramming cultures reduces reprogramming. A) Schematic representation of the experimental design for data in (B). NSC reprogramming cultures were treated daily with the DNA nuclease benzonase from the pre-iPS stage until the end of reprogramming. B) Flow cytometry-based quantification of GFP-expressing cells at the end of reprogramming, in cultures treated with different amounts of benzonase in the presence or absence of its co-factor MgCl₂.

FIG. 12: Removal of extracellular histones with small polyanions inhibits reprogramming. A) Schematic representation of the experimental design for data in (B). NSC reprogramming cultures were treated daily with the small polyanions MTS from the pre-iPS stage until the end of reprogramming. B) Schematic representation of the blocking of histones by MTS. C) Quantification of Oct4-GFP-expressing cells at the end of reprogramming, in cultures treated with MTS or control where no MTS were added.

FIG. 13: The transcription factor c-Myc is sufficient to induce expression and activation of PADI4 and release of extracellular citrullinated chromatin. A) Immunoblot analysis of PADI4 and citrullinated histone H3 (H3Cit) in neural stem cells transduced with c-Myc expressing virus, or empty vector (Mock), showing that transduction with c-Myc is sufficient to induce expression and activation of PADI4. Gapdh presented as loading control. B) Conditioned medium from cultures in A, showing that citrullinated histone H3 (H3Cit) is released to the extracellular space. d6 and d8 represent reprogramming day time points.

FIG. 14: Extracellular citrullinated histones interact with cell surface receptor Toll-like Receptor 2 (TLR2). Western blot analysis for H3Cit and TLR2 following immunoprecipitation with an anti-citrullinated histone H3 (H3CitR2) antibody or an anti-TLR2 antibody. WCE=whole cell extracts (input). Ctr=IgG control.

FIG. 15: Immunohistochemistry analysis of H3Cit in a model of tissue regeneration after Dextran Sulfate Sodium (DSS)-induced colitis. H3Cit (red; right panels in main figure and top panel in inset) is shown in mouse colon before (No insult) and at different times after 2% DSS treatment (Days Post Insult, DPI). E-cadherin (green; shown in left panels of main figure) marks the epithelium. DAPI (blue, shown in right panels and middle panels of main figure) marks cell nuclei. The inset shows that H3Cit is associated with extracellular NET-like structures.

FIG. 16: PADI4 and Ly6a are expressed in non-reprogramming cells. A) Fluorescence Activated Cell Sorting of reprogramming cultures based on the presence of GFP. GFP is expressed under the control of endogenous Oct4 regulatory elements and marks the reprogramming (iPS) cells (Theunissen et al. (2011) Current Biology, 21(1): 65-71, doi: https://doi.org/10.1016/j.cub.2010.11.074). B) Single cell RNA-sequencing analysis of reprogramming cultures, showing that Padi4 expression associates with Ly6a expression and the two are mutually exclusive with the iPS marker Nanog.

FIG. 17: Immunohistochemistry analysis of H3Cit and Ly6a at different phases of a DSS-colitis regeneration experiment. Ly6a (red) and H3Cit (green) are shown in mouse colon before (No insult), during (2% DSS, 1 week) and after (2% DSS 1 week+1 week recovery) the regeneration phase. E-cadherin (yellow) marks the epithelium. DAPI (blue) marks cell nuclei.

### DETAILED DESCRIPTION OF THE INVENTION

The present invention is based on the surprising finding by the inventors herein that the non-iPSCs within reprogramming cultures (i.e. cells other than the somatic cell to be reprogrammed), which also undergo a form of cell identity and acquire new cellular features and functions, play a role in supporting iPSC reprogramming (i.e. the somatic cell to be reprogrammed) through the release or secretion of pluripotency-promoting factors, i.e. that somatic cell reprogramming can be modulated non-cell autonomously, such as by using a reprogramming factor which derives from a cell other than the somatic cell to be reprogrammed. Such factors are sensed by, and help establish pluripotency within, the emerging iPSCs. The cells other than the somatic cell to be reprogrammed include non-reprogramming cells and so-called ‘bystander cells’, for example in a reprogramming culture. Therefore, as demonstrated herein, non-reprogramming cells in culture (or a subset thereof) do not merely fail to reprogramme as previously thought, but have an active role in promoting reprogramming, i.e. they are ‘active bystanders’. These findings open the possibility of harnessing non-cell-autonomous reprogramming factors to enhance iPSC generation in vitro or promote cell reprogramming (such as cell rejuvenation or tissue regeneration) in vivo. Furthermore, the identification of such non-cell autonomous reprogramming factors may allow their isolation or synthesis and administration to biological systems with the ultimate goal of achieving highly efficient reprogramming while minimising the direct genetic manipulation of somatic cells by transduction of reprogramming factors (such as some of the Yamanaka factors), which can predispose to cancerous transformation and teratoma formation. Thus the findings herein may be applied towards the achievement of efficient and safe reprogramming technologies in vitro and in vivo, impacting the areas of iPSC reprogramming and tissue repair, regeneration and rejuvenation.

According to a first aspect of the invention, there is provided a method of non-cell autonomous modulation of reprogramming, comprising providing a non-cell autonomous reprogramming factor and a somatic cell to be reprogrammed, wherein said non-cell autonomous reprogramming factor derives from a cell other than the somatic cell to be reprogrammed.

During reprogramming, somatic cells are converted or de-differentiated into pluripotent stem cells, i.e. they are induced to become pluripotent. The resulting reprogrammed cells are therefore known as induced pluripotent stem cells (iPSCs). Such iPSCs are similar to natural pluripotent stem cells (e.g. embryonic stem (ES) cells) in many respects, including in their ability to differentiate into multiple cell types and lineages, such as all types of cell found in an organism. iPSCs are forced to express genes and factors important for inducing and maintaining an ES cell-like state during reprogramming, and these derive from the reprogramming cell itself, often expressed by the reprogramming cell from the endogenous genes or from transfected/transduced genetic material encoding said factors, i.e. they are cell autonomous reprogramming factors. They include the Yamanaka factors OCT3/4, SOX2, KLF4 and c-MYC. NANOG and LIN28 may also be used together with the Yamanaka factors and can increase the induction of pluripotency.

References herein to one or more “cell autonomous reprogramming factors” or “autonomous reprogramming factors” include the Yamanaka factors, which include one or more of: OCT4, KLF4, c-MYC and SOX2. Thus in one embodiment, the one or more cell autonomous reprogramming factors are one or more Yamanaka factors. In a further embodiment, said one or more Yamanaka factors may additionally comprise LIN28 and NANOG. In an alternative embodiment, the one or more Yamanaka factors may be selected from one or more, two or more, three or more, four or more, five or more, six or more, seven or more, eight or more, or all of: OCT4, KLF4, c-MYC, SOX2, LIN28, NANOG, ESSRRB, NR5A2 and/or C/EBPα. In a further embodiment, the one or more Yamanaka factors are selected from: OCT4, KLF4, c-MYC and/or SOX2. In a yet further embodiment, the one or more Yamanaka factors are selected from: OCT4, KLF4, c-MYC, SOX2, LIN28 and/or NANOG. In still further embodiments, the one or more Yamanaka factors do not include c-MYC.

In certain embodiments, one or more cell autonomous reprogramming factors (e.g. Yamanaka factors) are provided which derive from the somatic cell to be reprogrammed. In a further embodiment, the one or more cell autonomous reprogramming factors derive from the reprogramming cell (e.g. the somatic cell to be reprogrammed). Thus in a yet further embodiment, the effect of the one or more cell autonomous reprogramming factors on the reprogramming cell (e.g. the somatic cell to be reprogrammed) is enhanced by the non-cell autonomous reprogramming factor defined herein. In another embodiment, the non-cell autonomous reprogramming factor enhances the reprogramming effect of the one or more cell autonomous reprogramming factors (e.g. Yamanaka factors). In a further embodiment, the non-cell autonomous reprogramming factor supplements or replaces one or more of the cell autonomous reprogramming factors. For example, the non-cell autonomous programming factor may replace the requirement for the cell autonomous reprogramming factor c-MYC, a potent oncogene, in the reprogramming cell (e.g. somatic cell to be reprogrammed). In a particular embodiment, the method is performed in a reprogramming culture comprising one or more cell autonomous reprogramming factors as defined herein (e.g. Yamanaka factors). In a yet further embodiment, the reprogramming culture comprising one or more cell autonomous reprogramming factors is in vitro. In one embodiment, the one or more cell autonomous reprogramming factors are expressed by the reprogramming cell (e.g. the somatic cell to be reprogrammed). In a further embodiment, the one or more cell autonomous reprogramming factors are expressed from a nucleic acid sequence encoding said cell autonomous factors in the reprogramming cell, in particular an exogenous nucleic acid sequence. In some embodiments, the nucleic acid encoding the one or more cell autonomous reprogramming factors is transfected or transduced into the reprogramming cell. In certain embodiments, the one or more cell autonomous reprogramming factors are Yamanaka factors as described hereinbefore. In a further embodiment, the Yamanaka factors are selected from one or more of: OCT4, KLF4, c-MYC, SOX2, LIN28, NANOG, ESSRRB, NR5A2 and/or C/EBPα, in particular one or more of: OCT4, KLF4, c-MYC, SOX2.

References herein to “somatic” refer to any type of cell that makes up the body of an organism, excluding germ cells and undifferentiated stem cells. Somatic cells therefore include, for example, fibroblasts, skin, heart, muscle, gut, eye, bone or blood cells, neurons and cells of the brain, peripheral and/or central nervous system, as well as their partly-differentiated tissue stem cells. In one embodiment, the methods of non-cell autonomous modulation of reprogramming defined herein comprise providing a somatic cell to be reprogrammed, such as to a pluripotent state (e.g. to an iPSC) as described herein.

### Non-Cell Autonomous Modulation of Reprogramming

References herein to “non-cell autonomous” refer to effects and processes that derive from cells other than those being manipulated or provided with a cell autonomous reprogramming factor (e.g. Yamanaka factors) described herein. For example, non-cell autonomous modulation as described herein comprises a reprogramming factor which effects/brings about reprogramming in a somatic cell but derives from a cell other than said somatic cell to be reprogrammed, i.e. the reprogramming factor is non-cell autonomous. In other words, a non-cell autonomous process is a process which occurs in one cell and brings about a change in another, distinct cell, while a non-cell autonomous factor is a factor released by one cell and which brings about a change in another, distinct cell. Thus references herein to a non-cell autonomous reprogramming factor that “derives from” a non-reprogramming/bystander cell include said factors that are “produced by”, “released from/by” and/or “secreted from/by” non-reprogramming/bystander cells, and the terms “derives from”, “produced by”, “released from/by” and “secreted from/by” may be used interchangeably herein. References herein to such non-cell autonomous reprogramming factors that “derive from” a non-reprogramming/bystander cell may also include analogues thereof (e.g. synthetic analogues) which perform the same function and can be considered as derivatives of said non-cell autonomous reprogramming factor. A further example of a non-cell autonomous process is where the differentiation state (e.g. the state of reprogramming) of a cell causes another cell of a different differentiation state to display an altered phenotype, such as because of cell-cell interactions or the release of signalling molecules. Thus, references herein to “non-cell autonomous reprogramming factor” refer to a factor which brings about reprogramming in a somatic cell but which derives from cells other than said somatic cell to be reprogrammed, such as non-reprogramming or bystander cells in vivo or in an in vitro culture. In certain embodiments, the non-cell autonomous reprogramming factor does not derive from the somatic cell to be reprogrammed. In a further embodiment, the non-cell autonomous reprogramming factor does not derive from the reprogramming cell.

Therefore in one embodiment, the methods of non-cell autonomous modulation of reprogramming defined herein comprise providing a non-cell autonomous reprogramming factor. Said non-cell autonomous reprogramming factor brings about or effects the reprogramming of a somatic cell but derives from a cell other than said somatic cell to be reprogrammed, such as wherein the non-cell autonomous reprogramming factor is a component of (e.g. is within or on the cell surface of) the non-reprogramming or bystander cell. In a particular embodiment, the non-cell autonomous reprogramming factor is released by or from the non-reprogramming or bystander cell. Thus in certain embodiments, the somatic cell to be reprogrammed is distinct from the cell other than said somatic cell or non-reprogramming/bystander cell.

In one embodiment, the non-cell autonomous reprogramming factor derives from a non-reprogramming cell. In a further embodiment, the non-cell autonomous reprogramming factor is released by or from a non-reprogramming cell. Such non-reprogramming cells include those in a reprogramming culture which appear to have failed to reprogramme. However, contrary to this apparent ‘failure’ to reprogramme, the inventors have surprisingly shown herein that these non-reprogramming cells (i.e. the bystander cells or cells other than the somatic cell to be reprogrammed) provide active support to reprogramming cells in culture in the form of a non-cell autonomous factor and that they may therefore be considered ‘active bystanders’. In a further embodiment, the non-cell autonomous reprogramming factor derives from a bystander cell. In a yet further embodiment, the non-cell autonomous reprogramming factor is released by or from a bystander cell. Thus, the terms “cell other than the somatic cell to be reprogrammed”, “non-reprogramming cell” and “bystander cell” may be used interchangeably herein and refer to any cell in vivo or in vitro (e.g. in culture) from which the non-cell autonomous reprogramming factor may derive, such as is released/secreted by or from.

References herein to “culturing” include the addition of cells (e.g. both the somatic cells to be reprogrammed and the non-reprogramming/bystander cell), to media comprising growth factors and/or essential nutrients. It will be appreciated that such culture conditions may be adapted as appropriate for the reprogramming of a somatic cell to an iPSC or iPSC-like state.

Thus in one embodiment, the method defined herein is performed in a culture. In a further embodiment, the method is performed in vitro. In a particular embodiment, the method is performed in an in vitro culture. In a further particular embodiment, the culture is a reprogramming culture comprising the somatic cell to be reprogrammed and one or more cells other than the somatic cell to be reprogrammed as described herein. Optionally, the non-cell autonomous reprogramming factor is provided to the reprogramming culture. Thus in a yet further particular embodiment, the culture is a reprogramming culture comprising the somatic cell to be reprogrammed and the non-cell autonomous reprogramming factor is provided to the reprogramming culture. In a still further embodiment, the method is performed in vitro and the non-cell autonomous reprogramming factor is provided to a reprogramming culture. In another embodiment, the method is performed in a reprogramming culture (e.g. in vitro) and the non-cell autonomous reprogramming factor is provided to said culture comprising the somatic cell to be reprogrammed and one or more cell other than the somatic cell to be reprogrammed. In yet other embodiment, the method is performed in a reprogramming culture (e.g. in vitro) and the non-cell autonomous reprogramming factor is provided to said culture comprising the somatic cell to be reprogrammed.

### Non-Cell Autonomous Reprogramming Factor

In certain embodiments of the present invention, the non-cell autonomous reprogramming factor is released from the non-reprogramming cell and/or a bystander cell as described herein. In a particular embodiment, the non-cell autonomous reprogramming factor is secreted from the non-reprogramming cell and/or a bystander cell.

As will be appreciated from the disclosures hereinbefore, the non-cell autonomous reprogramming factor released from the non-reprogramming/bystander cell in a reprogramming culture may be in response to cell autonomous reprogramming factors acting on said cell. The released or secreted non-cell autonomous reprogramming factor in turn acts on the somatic cell to be reprogrammed to bring about or effect reprogramming or the enhancement/promotion of reprogramming. Said non-cell autonomous reprogramming factor therefore acts on the somatic cell to be reprogrammed together with the one or more cell autonomous reprogramming factors (e.g. Yamanaka factors) described hereinbefore. Thus in one embodiment, wherein the method is performed in an in vitro reprogramming culture, the non-cell autonomous reprogramming factor is released into the culture medium. In a further embodiment, the culture medium comprising the non-cell autonomous reprogramming factor released by the non-reprogramming/bystander cell may be added to a somatic cell to be reprogrammed, optionally together with one or more cell autonomous reprogramming factors (e.g. Yamanaka factors) which derive from said somatic cell directly.

Thus in one aspect of the invention, there is provided a culture medium comprising the non-cell autonomous reprogramming factor released or secreted by the non-reprogramming or bystander cell. In one embodiment, the non-cell autonomous reprogramming factor is released into the culture medium by the non-reprogramming/bystander cell in response to one or more cell autonomous reprogramming factors acting on said cell. In a further embodiment, the cell culture medium optionally additionally comprises one or more cell autonomous reprogramming factors as described herein. As demonstrated herein, medium removed from reprogramming cultures in which cell autonomous reprogramming factors as described herein have been used (in particular the Yamanaka factors OCT4, KLF4, c-MYC and SOX2), i.e. a conditioned medium, is able to promote/enhance the reprogramming of somatic cells in a separate reprogramming culture. In one embodiment, the separate reprogramming culture to which the conditioned medium is added is at an earlier stage of reprogramming (e.g. is at an earlier timepoint of reprogramming) than the culture from which the medium is removed. In another aspect of the invention, there is provided a use of said culture medium (i.e. the conditioned culture medium) in a reprogramming culture. In some embodiments, said culture medium further comprises one or more cell autonomous reprogramming factors (e.g. Yamanaka factors) which derive directly from a somatic cell to be reprogrammed as described herein. In a yet further aspect, there is provided a kit for reprogramming a somatic cell, said kit comprising a culture medium comprising the non-cell autonomous reprogramming factor released or secreted by the non-reprogramming or bystander cell, optionally wherein said non-cell autonomous reprogramming factor has been released by the non-reprogramming/bystander cell in response to one or more cell autonomous reprogramming factors. In one embodiment, the kit further comprises and/or the cell medium of the kit further comprises one or more cell autonomous reprogramming factors (e.g. Yamanaka factors) which derive directly from the somatic cell to be reprogrammed as described herein, such as wherein said cell autonomous reprogramming factors are comprised in the form of nucleic acid sequences encoding said cell autonomous reprogramming factors.

In one embodiment, the non-cell autonomous reprogramming factor is chromatin or a component thereof. Thus in certain embodiments, the non-cell autonomous reprogramming factor that acts non-autonomously on the somatic cell to be reprogrammed and which effects reprogramming or promotes/enhances reprogramming of said somatic cell is extracellular chromatin. In a further embodiment, the non-cell autonomous reprogramming factor is an extracellular chromatin-associated moiety, such as an extracellular chromatin-associated nuclear protein. For example, in response to one or more cell autonomous reprogramming factors (e.g. Yamanaka factors), the non-reprogramming/bystander cells release or secrete chromatin (and/or a chromatin-associated moiety, such as an associated nuclear protein), such as into the culture medium. In another example, the non-reprogramming/bystander cells release or secrete chromatin in response to a known chromatin-release factor or as part of a known chromatin-release process, such as PADI4 activity. Components of chromatin include, without limitation, DNA, histones and/or nucleosomes containing histones. Thus in one embodiment, the non-cell autonomous reprogramming factor released by the non-reprogramming/bystander cell is extracellular DNA. In a further embodiment, the non-cell autonomous reprogramming factor is an extracellular nucleosome. In a yet further embodiment, the non-cell autonomous reprogramming factor released by the non-reprogramming/bystander cell is a histone as described hereinbefore. In a still further embodiment, the non-cell autonomous reprogramming factor is histone H3. In some embodiments, the chromatin, such as the histone, is modified such as post-translationally modified. Modifications include phosphorylation, ubiquitination, SUMOylation, citrullination and ADP-ribosylation, in particular citrullination. Thus in a further embodiment, the signal or signalling agent is a citrullinated histone. In a particular embodiment, the signal or signalling agent is citrullinated histone H3 (H3Cit). As has been demonstrated herein, extracellular chromatin, in particular containing citrullinated histone H3, can be detected in the culture medium of reprogramming cultures. Those cells within the reprogramming culture which can be identified by staining for citrullinated histone H3 are distinct from those undergoing reprogramming (as identified using OCT4 and NANOG expression), i.e. cells which release/secrete the non-cell autonomous reprogramming factor and thus stain positive for citrullinated histone H3 are the non-reprogramming/bystander cells. As also demonstrated herein, the blocking of chromatin sensing pathways in the somatic cell to be reprogrammed (in particular the cGAS/STING and TLR pathways) reduces the enhancing/promoting effect on reprogramming in a reprogramming culture when a conditioned medium as described herein is added.

Thus in another aspect of the invention, there is provided a use of an agonist or activator of a chromatin sensing pathway in an in vitro method of reprogramming a somatic cell. In a further aspect, there is provided an agonist or activator of a chromatin sensing pathway for use in a method of treating and/or ameliorating a degenerative disease or disorder or for use in the rejuvenation, repair or regeneration of a tissue or organ, wherein said method comprises reprogramming a somatic cell in vivo. In another aspect, there is provided an agonist or activator of a chromatin sensing pathway for use in a method of rejuvenating, repairing or regenerating a tissue or organ. In a further aspect, there is provided an agonist or activator of a chromatin sensing pathway for use in a method of rejuvenating, repairing or regenerating a tissue or organ, wherein said method comprises reprogramming a somatic cell in vivo. In a yet further aspect, there is provided a method of rejuvenating, repairing or regenerating a tissue or organ, said method comprising the method of non-cell autonomous modulation of reprogramming defined herein, and said method further comprising administering to a subject an agonist or activator of a chromatin sensing pathway. In a still further aspect, there is provided a method of rejuvenating a tissue or organ, said method comprising administering to a subject an agonist or activator of a chromatin sensing pathway, and said method comprises reprogramming a somatic cell in vivo. In some embodiments, the chromatin sensing pathway agonist/activator is comprised in a pharmaceutical composition, optionally further comprising one or more pharmaceutically acceptable carriers, diluents and/or excipients. Thus in a yet further aspect of the invention, there is provided a pharmaceutical composition comprising an agonist or activator of a chromatin sensing pathway for use in a method of treating and/or ameliorating a degenerative disease or disorder, or for use in the rejuvenation, repair or regeneration of a tissue or organ.

Histones are highly basic proteins abundant in lysine and arginine residues that are found in eukaryotic cell nuclei. They act as spools around which DNA winds to create structural units called nucleosomes, which are in turn are wrapped into 30 nm fibres that form tightly packed chromatin. Histones play important roles in gene regulation and DNA replication. The five families of histones are designated H1/H5 (linker histones), H2, H3 and H4 (core histones). The nucleosome core is formed of two H2A-H2B dimers and two H3-H4 dimers. The tight wrapping of DNA around histones is to a large degree a result of electrostatic attraction between the positively charged histones and negatively charged phosphate backbone of DNA. The core histones (H2A, H2B, H3 and H4) are relatively similar in structure and are highly conserved through evolution, all featuring a ‘helix turn helix turn helix’ motif. They also share the feature of long ‘tails’ on one end of the amino acid structure—this being the location of post-translational modification. Histones may be chemically modified through the action of enzymes to regulate gene transcription. The most common modifications are the methylation of arginine or lysine residues or the acetylation of lysine. Methylation can affect how other proteins such as transcription factors interact with the nucleosomes, while lysine acetylation eliminates a positive charge on lysine thereby weakening the electrostatic attraction between histone and DNA, and resulting in partial unwinding of the DNA making it more accessible for gene expression. Further modifications include modifications of the tail include phosphorylation, ubiquitination, SUMOylation, citrullination and ADP-ribosylation. Citrullination (also known as peptidylarginine deimination) is the conversion of the amino acid arginine to citrulline. Citrulline is not one of the 20 standard amino acids encoded by DNA in the genetic code and is instead the result of a post-translational modification. Citrullination is distinct from the formation of the free amino acid citrulline as part of the urea cycle or as a by-product of enzymes of the nitric oxide synthase family.

Citrullination is catalysed by enzymes called arginine deiminases (ADIs) which catalyse the deimination of free arginine, while protein-arginine deiminases or peptidylarginine deiminases (PADIs or PADs) replace the primary ketimine group (>C═NH) by a ketone group (>C═O). Arginine is positively charged at a neutral pH, whereas citrulline has no net charge. This increases the hydrophobicity of the protein, which can lead to changes in protein folding, affecting the structure and function. The immune system can attack citrullinated self-proteins, leading to autoimmune diseases such as rheumatoid arthritis (RA) and multiple sclerosis (MS). The release of chromatin from cells is well known in the context of immune cells and inflammation, in particular in neutrophils which release chromatin/nucleic material (NETs; neutrophil extracellular traps) in a process called NETosis. One PAD enzyme, PADI4 has been shown to be involved in NETosis in immune cells, but this function in other cell types has not been previously described. Thus, the novel and surprising findings presented herein which demonstrate that reprogramming can be reduced/inhibited by PADI4 inhibition/knock-down, suggest that the non-cell autonomous reprogramming factor released from non-reprogramming/bystander cells as defined herein may be chromatin, in particular extracellular chromatin comprising citrullinated histone H3. The additional findings herein that the inhibition/blocking of chromatin/DNA sensing pathways also reduces/inhibits reprogramming further supports this suggestion.

### In Vitro, in Vivo and Therapeutic Uses

In one aspect of the invention, there is provided a use of the non-cell autonomous reprogramming factor defined herein, or an analogue thereof, in an in vitro method of reprogramming a somatic cell. As defined hereinbefore, in one embodiment the non-cell autonomous reprogramming factor is chromatin or a component thereof, such as extracellular chromatin. In a further embodiment, the non-cell autonomous reprogramming factor is a nucleosome. In another embodiment, the non-cell autonomous reprogramming factor is extracellular DNA. In a further embodiment, the non-cell autonomous reprogramming factor is a histone. In a yet further embodiment, the non-cell autonomous reprogramming factor is histone H3. In a particular embodiment, the non-cell autonomous reprogramming factor is citrullinated histone H3. In a further embodiment, the in vitro method of reprogramming a somatic cell is a reprogramming culture as defined hereinbefore.

In one embodiment, the non-cell autonomous reprogramming factor is isolated, such as purified, from reprogramming cultures comprising non-reprogramming or bystander cells. In another embodiment, the non-cell autonomous reprogramming factor may be synthetic. In a further embodiment, the non-cell autonomous reprogramming factor has been generated in vitro, such as wherein the non-cell autonomous reprogramming factor is a histone, in particular histone H3, which has been citrullinated using a PAD enzyme in vitro. In another embodiment, in vitro generation of the non-cell autonomous reprogramming factor comprises in vitro synthesis, such as in vitro synthesis of a histone, in particular histone H3, containing one or more citrulline residues in place of one or more arginine residues. In a yet further embodiment, the non-cell autonomous reprogramming factor is a functional analogue of the non-cell autonomous reprogramming factor defined herein, such as a functional analogue of chromatin, extracellular DNA or a histone, such as of histone H3, in particular of citrullinated histone H3. Such analogues will be readily appreciated to include those which share significant structural similarity with the signal or signalling agent. However, analogues which do not share structural similarity but which perform similar functions, e.g. activate the same or similar (such as redundant) signalling pathways, are also within the scope of the term “analogues” herein.

Thus in one embodiment, the non-cell autonomous reprogramming factor is an agonist of a chromatin sensing pathway. In a further embodiment, the non-cell autonomous reprogramming factor is a cGAS/STING pathway agonist. In another embodiment, the non-cell autonomous reprogramming factor is a TLR agonist. In certain embodiments, the non-cell autonomous reprogramming factor is an agonist of the TLR2, TLR3 and/or TLR4 pathways, in particular a TLR2 agonist as demonstrated herein, an agonist of the TLR3 pathway which has been shown to sense double stranded DNA, or an agonist of the TLR4 pathway which senses citrullinated histones. In a yet further embodiment, the non-cell autonomous reprogramming factor is an agonist of the extracellular chromatin receptor, CCDC25 (as described in Yang et al. (2020) Nature, 583:133-138, doi: https://doi.org/10.1038/s41586-020-2394-6).

In some embodiments, the methods described herein may be performed in vivo. Thus in a further aspect of the invention, there is provided the non-cell autonomous reprogramming factor defined herein for use in an in vivo method of non-cell autonomous modulation of reprogramming. In another aspect, there is provided an in vivo method of non-cell autonomous modulation of reprogramming, said method comprising administering the non-cell autonomous reprogramming factor defined herein to a subject. Thus in certain embodiments, the in vivo methods of non-cell autonomous reprogramming comprise administering the non-cell autonomous reprogramming factor, or an analogue thereof, to a subject. Such administration may be systemically, e.g. enteral or parenteral, such as via intravenous infusion, or locally, such as directly into the tissue or organ to be treated/rejuvenated, e.g. by topical administration.

In a further aspect, there is provided the non-cell autonomous reprogramming factor defined herein, or an analogue thereof, for use in a method of treating and/or ameliorating a degenerative disease or disorder. In another aspect, there is provided the non-cell autonomous reprogramming factor defined herein, or an analogue thereof, for use in a method of treating and/or ameliorating a degenerative disease or disorder, wherein said method comprises reprogramming a somatic cell in vivo. Thus in certain embodiments, the subject is suffering or is at risk of suffering from a degenerative disease or disorder. In a further aspect, there is provided a method of treating and/or ameliorating a degenerative disease or disorder, said method comprising the method of non-cell autonomous modulation of reprogramming defined herein, and said method further comprising administering to a subject the non-cell autonomous reprogramming factor defined herein, or an analogue thereof. In a yet further aspect, there is provided a method of treating and/or ameliorating a degenerative disease or disorder, said method comprising administering to a subject the non-cell autonomous reprogramming factor defined herein, and said method comprises reprogramming a somatic cell in vivo. In one embodiment, the subject is suffering or is at risk of suffering from a degenerative disease or disorder of the skin. In an alternative embodiment, the subject is suffering or is at risk of suffering from a degenerative disease or disorder of the pancreas, such as type 2 diabetes. In a further embodiment, the subject is suffering or is at risk of suffering from a neurodegenerative disorder. In a yet further embodiment, the subject is suffering or is at risk of suffering from a disease or disorder of the blood and/or bone marrow. In a still further embodiment, the subject is suffering or is at risk of suffering from a disease or disorder of the heart. Thus, in one embodiment, the disease or disorder is cardiovascular disease. In a further embodiment, the disease or disorder is a cardiomyopathy. In another embodiment, the disease or disorder is ischaemic heart disease. In a yet further embodiment, the disease or disorder is cardiac arrhythmia. In another embodiment, the disease or disorder is heart failure. In another embodiment, the subject is suffering or is at risk of suffering from a disease or disorder of the gut. In a yet further embodiment, the subject is suffering or is at risk of suffering from a disease or disorder of the eye.

In another embodiment, the subject is suffering or is at risk of suffering from a degenerative disease or disorder of the brain, central and/or peripheral nervous system, in particular the brain or central nervous system. For example, the subject may be suffering from a neurodegenerative disease or disorder which may affect the brain, the central or the peripheral nervous system. In particular the role of the pluripotency factor c-MYC in stem cells of the central nervous system (oligodendrocyte progenitor cells) has previously been demonstrated, in which its expression drives the functional rejuvenation of these cells and inhibition leads to an aged-like phenotype (Neumann et al. (2021) Nature Aging, 1:826-837, doi: https://doi.org/10.1038/s43587-021-00109-4). Thus, the findings herein that the non-cell autonomous reprogramming factor is induced by one or more of the cell autonomous reprogramming factors, in particular the cell autonomous reprogramming factor c-MYC, and may therefore be used to substitute for cell autonomous reprogramming factors, demonstrate the potential for the non-cell autonomous reprogramming factor in treatment of diseases and disorders of the brain, central and/or peripheral nervous system, as well as the rejuvenation, regeneration and/or repair of said tissues and their cells.

In a further embodiment, the subject is suffering or is at risk of suffering from damage to the brain, central and/or peripheral nervous system following injury. Examples of injuries which can lead to damage of the brain, central and/or peripheral nervous system include, without limitation, ischemic brain injury, traumatic brain injury, hypoxia, tumours of the brain or nervous systems, infections, surgery and poisoning of the brain or nervous systems. In particular it has previously been demonstrated that the release of phospholipase PLA2G2E following ischemic brain injury leads to the activation of PADI4 through the generation of dihomo-γ-linolenic acid (DGLA), and that this triggers neural repair by activating the transcription of genes associated with recovery processes after ischemic stroke (Nakamura et al. (2023) Neuron, 111(19): 2995-3010, doi: https://doi.org/10.1016/j.neuron.2023.06.024). Thus, the findings herein that propose PADI4-mediated extracellular signals, such as extracellular chromatin, DNA and/or citrullinated histone H3, as the non-cell autonomous reprogramming factor demonstrate the potential utility in treating damage to the brain, central and/or peripheral nervous systems or in their rejuvenation, regeneration and/or repair following injury.

In another embodiment, the methods described herein are for the rejuvenation of a tissue or organ. Rejuvenation is useful in the reversal of the effects of ageing on said tissue or organ. Thus in some embodiments, the tissue or organ is aged, such as is obtained from an aged subject or is in an aged subject. Thus in another aspect of the invention, there is provided the non-cell autonomous reprogramming factor defined herein, or an analogue thereof, for use in a method of rejuvenating a tissue or organ. In a further aspect, there is provided the non-cell autonomous reprogramming factor defined herein, or an analogue thereof, for use in a method of rejuvenating a tissue or organ, wherein said method comprises reprogramming a somatic cell in vivo. In a yet further aspect, there is provided a method of rejuvenating a tissue or organ, said method comprising the method of non-cell autonomous modulation of reprogramming defined herein, and said method further comprising administering to a subject the non-cell autonomous reprogramming factor defined herein, or an analogue thereof. In a still further aspect, there is provided a method of rejuvenating a tissue or organ, said method comprising administering to a subject the non-cell autonomous reprogramming factor defined herein, and said method comprises reprogramming a somatic cell in vivo. In certain embodiments, the method of rejuvenating a tissue or organ comprises reprogramming a somatic cell according to the methods defined herein and providing said reprogrammed somatic cell to a subject in need thereof. In a further embodiment, the somatic cell to be reprogrammed may be derived from the subject in need of treatment and/or amelioration of a degenerative disease or disorder, or from the subject in need of tissue or organ rejuvenation. Thus in some embodiments, the methods described herein are performed ex vivo.

In another embodiment, the methods described herein are for the regeneration or repair of a tissue or organ. Regeneration and/or repair of tissue or organ may be required in response or following damage, such as damage due to an acute injury or disease, or due to a chronic disease or disorder. Damage may also occur in aged tissues and organs. Thus in some embodiments, the tissue or organ is damaged, such as damaged as a result of an acute or chronic disease or disorder. In other embodiments, the damaged tissue or organ is aged, such as is obtained from an aged subject or is in an aged subject. Thus in another aspect of the invention, there is provided the non-cell autonomous reprogramming factor defined herein, or an analogue thereof, for use in a method of regenerating or repairing a tissue or organ. In a further aspect, there is provided the non-cell autonomous reprogramming factor defined herein, or an analogue thereof, for use in a method of regenerating or repairing a tissue or organ, wherein said method comprises reprogramming a somatic cell in vivo. In a yet further aspect, there is provided a method of regenerating or repairing a tissue or organ, said method comprising the method of non-cell autonomous modulation of reprogramming defined herein, and said method further comprising administering to a subject the non-cell autonomous reprogramming factor defined herein, or an analogue thereof. In a still further aspect, there is provided a method of regenerating or repairing a tissue or organ, said method comprising administering to a subject the non-cell autonomous reprogramming factor defined herein, and said method comprises reprogramming a somatic cell in vivo. In certain embodiments, the method of regenerating or repairing a tissue or organ comprises reprogramming a somatic cell according to the methods defined herein and providing said reprogrammed somatic cell to a subject in need thereof. In a further embodiment, the somatic cell to be reprogrammed may be derived from the subject in need of treatment and/or amelioration of a degenerative disease or disorder, or from the subject in need of tissue or organ regeneration or repair. Thus in some embodiments, the methods of non-cell autonomous modulation of reprogramming described herein are performed ex vivo.

In one embodiment the use of the non-cell autonomous reprogramming factor defined herein, or an analogue thereof, additionally comprises one or more cell autonomous reprogramming factors as described herein. In another embodiment, the in vitro, ex vivo or in vivo method of non-cell autonomous modulation of reprogramming additionally comprises one or more cell autonomous reprogramming factors as described herein. In a further embodiment, the one or more cell autonomous reprogramming factors comprise one or more Yamanaka factors as described herein. In a yet further embodiment, the Yamanaka factors are selected from one or more of: OCT4, KLF4, c-MYC, SOX2, LIN28, NANOG, ESSRRB, NR5A2 and/or C/EBPα, in particular one or more of: OCT4, KLF4, c-MYC, SOX2.

In some embodiments, the non-cell autonomous reprogramming factor is comprised in a pharmaceutical composition, optionally further comprising one or more pharmaceutically acceptable carriers, diluents and/or excipients. Thus in a yet further aspect of the invention, there is provided a pharmaceutical composition comprising the non-cell autonomous reprogramming factor for use in a method of treating and/or ameliorating a degenerative disease or disorder, or for use in the rejuvenation, regeneration or repair of a tissue or organ. In further aspects, there is provided a method of treating and/or ameliorating a degenerative disease or disorder or a method of rejuvenating, regenerating or repairing a tissue or organ, said method comprising administering to a subject the pharmaceutical composition comprising the non-cell autonomous reprogramming factor defined herein.

In a further aspect, there is provided a reprogrammed somatic cell obtainable by the method of non-cell autonomous modulation of reprogramming defined herein. In one aspect, there is provided a pharmaceutical composition comprising said reprogrammed somatic cell. In one embodiment, the pharmaceutical composition optionally further comprises one or more pharmaceutically acceptable carriers, diluents and/or excipients.

Thus in another aspect, there is provided the reprogrammed somatic cell obtained by the methods described herein or the pharmaceutical composition comprising said reprogrammed somatic cell for use in a method of treating and/or ameliorating a degenerative disease or disorder, or for use in a method of rejuvenating, regenerating or repairing a tissue or organ. In a further aspect, there is provided a method of treating and/or ameliorating a degenerative disease or disorder or a method of rejuvenating, regenerating or repairing a tissue or organ, said method comprising administering to a subject the reprogrammed somatic cell obtained by the methods described herein or the pharmaceutical composition comprising said reprogrammed somatic cell.

In some embodiments, the methods of reprogramming as described herein may comprise incomplete and/or partial reprogramming. Thus, references herein to “reprogramming” may be used interchangeably with “partial/incomplete reprogramming”. Such partial/incomplete reprogramming is compared to a cell with a high level of potency (e.g. an embryonic stem (ES) cell or an iPSC), in particular compared to an iPSC. During the process of iPSC reprogramming (i.e. full reprogramming), somatic cells are converted or de-differentiated into pluripotent stem cells. Such iPSCs are similar to natural pluripotent stem cells (e.g. ES cells) in many respects, including in their ability to differentiate into multiple cell types. However, during iPSC reprogramming, DNA methylation age is reset to zero years old regardless of the age of the donor tissue from which the somatic cell was obtained. As such, the process of iPSC reprogramming resets the epigenetic signature of the somatic cell to an embryonic-like state and causes loss of somatic cell lineage identity. Conversely partial/incomplete reprogramming does not completely reset the epigenetic signature of the reprogrammed cell and, for example, somatic lineage identity may be retained.

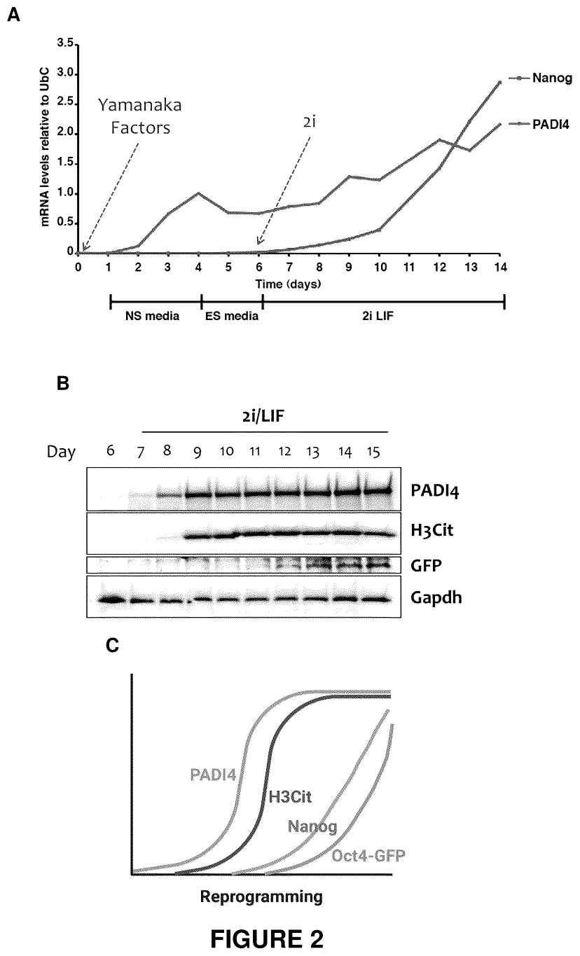

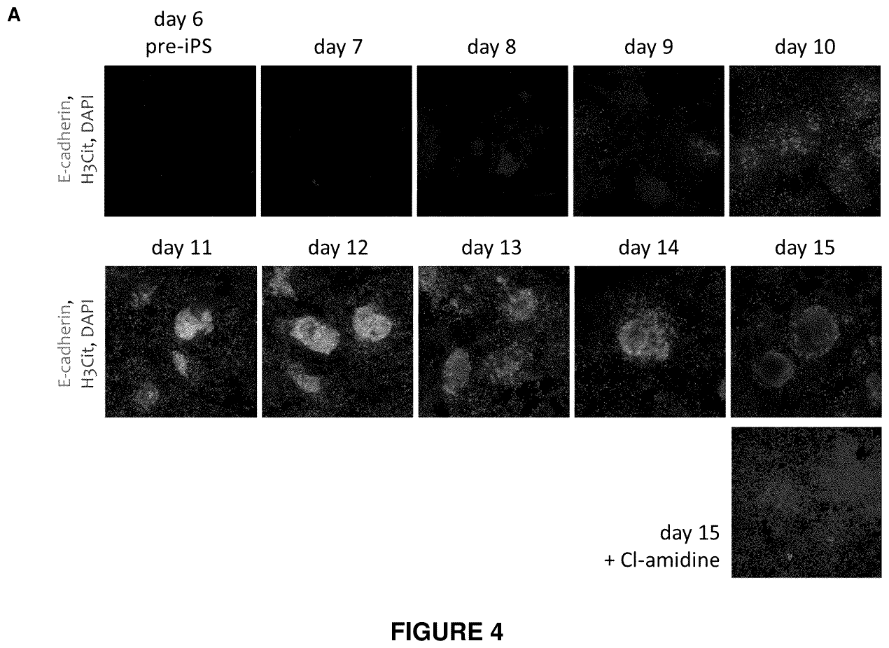

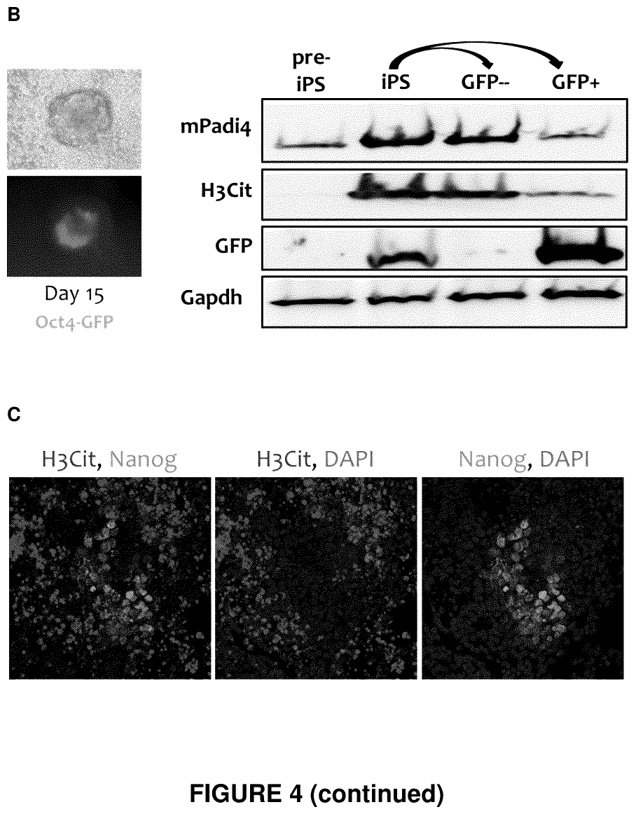

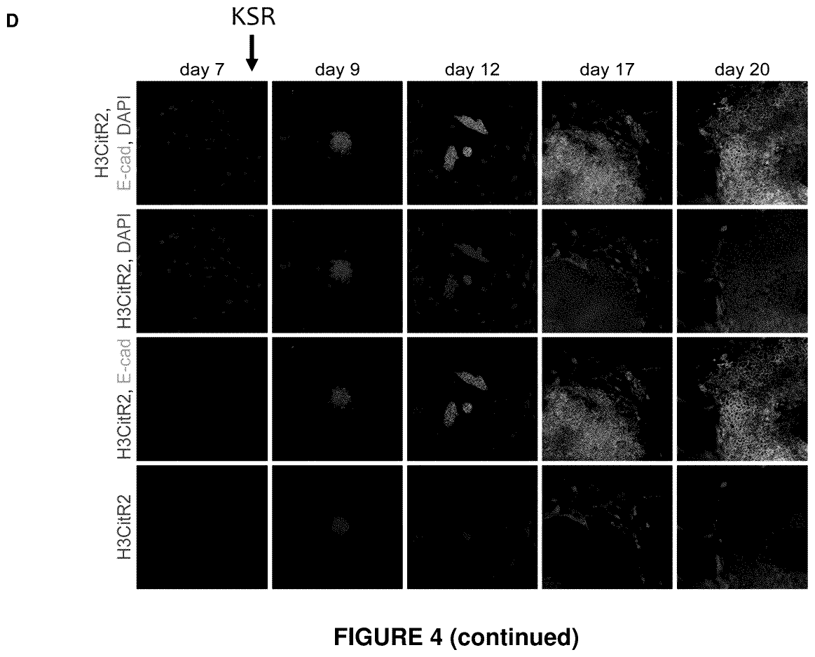

Such partial reprogramming will be appreciated to be applicable to the methods described herein in light of the data of at least Examples 1 and 4, and in FIGS. 2B, 2C and 4 herein that demonstrate the levels of PADI4 mRNA and protein levels increasing in reprogramming cultures prior to the expression of pluripotency genes. Also prior to the expression of pluripotency genes, the levels of citrullinated histone H3 (H3Cit; a non-cell autonomous reprogramming factor) are seen to increase. Thus, release of a non-cell autonomous reprogramming factor as defined herein precedes reprogramming of the somatic cell to be reprogrammed and/or is an early event in the process of reprogramming. As such, it will be readily appreciated that the methods described herein may be applied to partial/incomplete reprogramming which may require only the early stages/events of reprogramming to be performed/completed.

It will be appreciated that references herein to a patient or subject relate equally to animals and humans and that the invention finds particular utility in veterinary treatment of any of the above mentioned diseases, disorders and conditions which are also present in said animals.

It will also be appreciated that references herein to “treatment” and “amelioration” include such terms as “prevention”, “reversal” and “suppression”. Furthermore, such references include administration of the reprogrammed somatic cell or composition comprising the reprogrammed somatic cell as defined herein prior to the onset of the disease or disorder, e.g. wherein the subject is at risk of the disease or disorder. Administration of the reprogrammed somatic cell or composition as defined herein may also be anticipated after the induction event of the injury, damage, disease or disorder, either before clinical presentation of said disease or disorder, or after symptoms manifest. Such references further include performing the method of non-cell autonomous modulation of reprogramming as defined herein in vivo either prior to the onset of the disease or disorder, or after the induction event of the disease or disorder.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

Throughout the specification and the claims which follow, unless the context requires otherwise, the word “comprise”, and variations thereof such as “comprises” and “comprising”, will be understood to imply the inclusion of a stated integer, step, group of integers or group of steps but not to the exclusion of any other integer, step, group of integers or group of steps.

In addition, as used herein and the appended claims, the singular forms “a”, “an” and “the” include plural referents unless the content clearly dictates otherwise. Thus, for example reference to “a reprogrammed somatic cell” includes two or more such cells, or reference to “a non-reprogramming/bystander cell” includes two or more such non-reprogramming or bystander cells, i.e. two or more cells other than the somatic cell(s) to be reprogrammed and the like.

It will be understood that all embodiments described herein may be applied to all aspects of the invention and vice versa, and such combinations would be readily apparent from the description provided herein and to those skilled in the art.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art.

### CLAUSES

A set of clauses defining the invention, its aspects and embodiments is as follows:
1. A method of non-cell autonomous modulation of reprogramming, comprising providing a non-cell autonomous reprogramming factor and a somatic cell to be reprogrammed, wherein said non-cell autonomous reprogramming factor derives from a cell other than the somatic cell to be reprogrammed.
2. The method of clause 1, wherein the cell other than the somatic cell to be reprogrammed is a non-reprogramming cell, such as a bystander cell.
3. The method of clause 1 or clause 2, wherein the non-cell autonomous reprogramming factor derives from a non-reprogramming cell, such as a bystander cell.
4. The method of any one of clauses 1 to 3, wherein the non-cell autonomous reprogramming factor does not derive from the somatic cell to be reprogrammed, such as does not derive from the reprogramming cell.
5. The method of any one of clauses 1 to 4, wherein the non-cell autonomous reprogramming factor is released from the cell other than the somatic cell to be reprogrammed, such as is released from a non-reprogramming cell and/or a bystander cell.
6. The method of any one of clauses 1 to 5, wherein the non-cell autonomous reprogramming factor is secreted from the cell other than the somatic cell to be reprogrammed, such as is secreted from a non-reprogramming cell and/or a bystander cell.
7. The method of clause 5 or clause 6, wherein the method is performed in an in vitro reprogramming culture and the non-cell autonomous reprogramming factor is released or secreted into the culture medium.
8. The method of any one of clauses 1 to 7, wherein the non-cell autonomous reprogramming factor is chromatin or a component thereof, or a functional analogue thereof.
9. The method of clause 8, wherein the non-cell autonomous reprogramming factor is an extracellular nucleosome or a component thereof, or a functional analogue thereof.
10. The method of clause 8 or clause 9, wherein the non-cell autonomous reprogramming factor is extracellular DNA or a functional analogue thereof.
11. The method of any one of clauses 8 to 10, wherein the chromatin comprises histone H3 or a functional analogue thereof, such as wherein the non-cell autonomous reprogramming factor is histone H3.
12. The method of any one of clauses 8 to 11, wherein the chromatin or component thereof is modified, such as post-translationally modified.
13. The method of clause 12, wherein the chromatin or component thereof is citrullinated.
14. The method of clause 12 or clause 13, wherein the chromatin or component thereof comprises a citrullinated histone.
15. The method of clause 14, wherein the citrullinated histone is citrullinated histone H3.
16. The method of any one of clauses 1 to 15, wherein the non-cell autonomous reprogramming factor is an agonist of a chromatin sensing pathway.
17. The method of any one of clauses 1 to 16, wherein the method additionally comprises providing a non-reprogramming cell and/or a bystander cell.
18. The method of any one of clauses 1 to 17, wherein the method is performed in vitro, such as an in vitro reprogramming culture.
19. The method of any one of clauses 1 to 17, wherein the method is performed ex vivo, such as in an in vitro reprogramming culture.
20. The method of any of clauses 1 to 19, wherein one or more cell autonomous reprogramming factors are provided which derive from the somatic cell to be reprogrammed, such as the reprogramming cell.
21. The method of clause 20, wherein the one or more cell autonomous reprogramming factors are Yamanaka factors, such as a Yamanaka factor selected from one or more of: OCT4, KLF4, c-MYC, SOX2, LIN28, NANOG, ESSRRB, NR5A2 and/or C/EBPα, in particular one or more of: OCT4, KLF4, c-MYC and SOX2.
22. The method of clause 20 or clause 21, wherein the one or more cell autonomous reprogramming factors are expressed by the somatic cell to be reprogrammed, such as wherein the one or more cell autonomous reprogramming factors are expressed from a nucleic acid sequence encoding said cell autonomous factors in the somatic cell to be reprogrammed.
23. The method of any one of clauses 20 to 22, wherein the non-cell autonomous reprogramming factor enhances or promotes the reprogramming effect of the one or more cell autonomous reprogramming factors.
24. The method of any one of clauses 1 to 23, wherein reprogramming is full reprogramming or is partial and/or incomplete reprogramming.
25. The non-cell autonomous reprogramming factor defined in any one of clauses 3 to 16, or an analogue thereof, for use in a method of treating and/or ameliorating a degenerative disease or disorder or in a method of rejuvenating, regenerating or repairing a tissue or organ, wherein said method comprises reprogramming a somatic cell in vivo.
26. The non-cell autonomous reprogramming factor defined in any one of clauses 3 to 16, or an analogue thereof, for use in a method of treating and/or ameliorating a degenerative disease or disorder or in a method of rejuvenating, regenerating or repairing a tissue or organ, wherein said non-cell autonomous reprogramming factor derives from a cell other than the somatic cell to be reprogrammed in vivo.
27. The non-cell autonomous reprogramming factor, or analogue thereof, for use of clause 25 or clause 26, wherein the methods additionally comprise one or more cell autonomous reprogramming factors, such as Yamanaka factors, in particular a Yamanaka factor selected from one or more of: OCT4, KLF4, c-MYC, SOX2, LIN28, NANOG, ESSRRB, NR5A2 and/or C/EBPα, such as one or more of: OCT4, KLF4, c-MYC and SOX2.

The invention will now be described using the following, non-limiting examples:

### EXAMPLES

### Example 1: Induction of PADI4 Expression and Activity Precedes Reprogramming

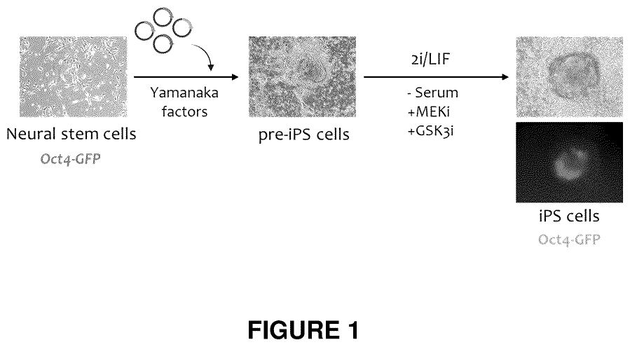

To investigate the timing of PADI4 expression during reprogramming, the method of reprogramming neural stem cells (NSCs) to iPSCs described in Theunissen et al. (2011) Current Biology, 21(1): 65-71 (doi: https://doi.org/10.1016/j.cub.2010.11.074) was used (FIG. 1). Yamanaka factors were transduced into the mouse NSC line, NSO4G, and the cells were cultured under standard reprogramming conditions. NSCs do not express Padi4 or Nanog prior to reprogramming. At various reprogramming timepoints, the reprogramming cells were harvested and the levels of mRNA encoding PADI4 and NANOG were quantified by qPCR (FIG. 2A) and protein levels (FIG. 2B).

As shown in FIGS. 2A and 2B, PADI4 mRNA and protein levels increase in reprogramming cultures prior to the expression of NANOG and OCT4, two pluripotency genes. Following the increase of PADI4 protein levels but also prior to the expression of NANOG and OCT4, the levels of citrullinated histone H3 (H3Cit; the product of PADI4 activity) increase (FIGS. 2B and 2C). Therefore, PADI4 expression and activity as measured by citrullinated histone H3 increases in reprogramming cultures prior to reprogramming.

### Example 2: Pharmacological or Genetic Inhibition of PADI4 Reduces Reprogramming

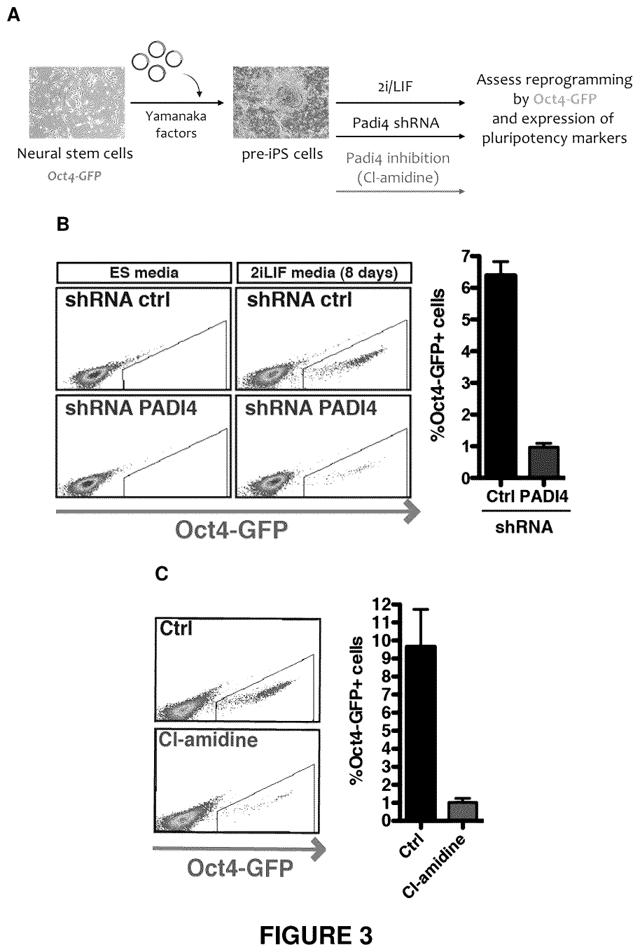

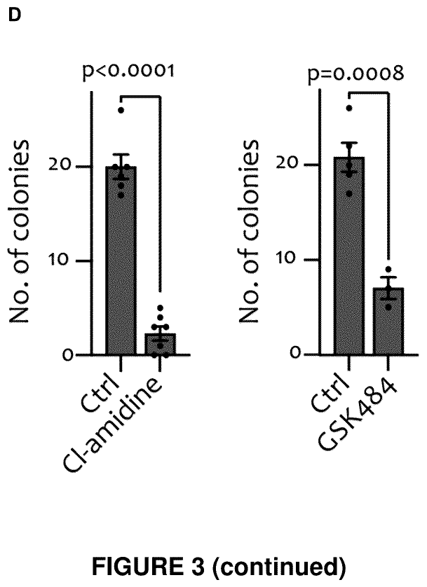

To determine if the expression and activity of PADI4 seen in the reprogramming cultures of Example 1 have a role in reprogramming, pharmacological inhibition by an established PADI4 inhibitor (CI-amidine) or genetic inhibition using PADI4-targetting short hairpin RNAa (shRNAa) were used in reprogramming cultures following the induction of reprogramming using Yamanaka factors (FIG. 3A).

As shown in FIG. 3B, the proportion of successfully reprogramming cells expressing OCT4-GFP was significantly reduced when a PADI4-targetting shRNA was added to the reprogramming cultures. The same result is achieved with the PADI4 inhibitor CI-amidine (FIG. 3C).

A different model of reprogramming, using human fibroblasts without the OCT4-GFP reporter also demonstrates these findings, with the number of colonies (representative of the number of successfully reprogrammed cells) significantly reduced when either CI-amidine or another established PADI4 inhibitor, GSK484, are added to the reprogramming culture (FIG. 3D).

Therefore, data from several independent and established PADI4 inhibitors/inactivators in two independent reprogramming culture systems show that inactivation of PADI4 activity reduces/inhibits reprogramming.

### Example 3: H3Cit-Positive Cells Surround the Emerging iPSC Colonies, and PADI4 and Histone Citrullination are in the Non-Reprogramming Cells

The reprogramming cultures of Example 1 were analysed for their expression of PADI4 and citrullinated histone H3 by microscopy. As shown in FIG. 4A, the cells staining positive for H3Cit are distinct from those which are reprogramming and are E-cadherin positive. Furthermore, if reprogramming cultures are sorted for those cells expressing OCT4-GFP (i.e. which are reprogramming; see FIG. 4B, left panel) and those which are OCT4-GFP negative (i.e. non-reprogramming cells), significant PADI4 and H3Cit protein levels are detectable in the non-reprogramming cells with very little in the OCT4-GFP positive reprogramming cells (FIG. 4B). Furthermore, H3Cit-positive cells are mutually exclusive with Nanog-positive iPS cells (FIG. 4C). The same results are seen in the non-OCT4-GFP expressing fibroblast reprogramming model (FIG. 4D).

The data presented herein shows that in both mouse neural stem cell and human fibroblast reprogramming the cells that express and activate PADI4 are mutually exclusive with the reprogramming cells/iPSCs. Therefore, in light of the fact that inhibition of PADI4 inhibits iPSC generation, this suggests that PADI4 is acting in a non-cell autonomous manner, i.e. that the PADI4-expressing cells are not merely failing to reprogramme, but have an active role in promoting reprogramming within the culture. It has also been shown using single-cell RNA sequencing (data not shown) that the non-reprogramming cells also change during the duration of the reprogramming culture, suggesting that they also undergo a form of identity change (a different kind of reprogramming). As such, they may be considered as “active bystanders” in the process of reprogramming, in particular in reprogramming cultures.

### Example 4: Medium Conditioned by Reprogramming Cultures Increases Reprogramming of Recipient Cells

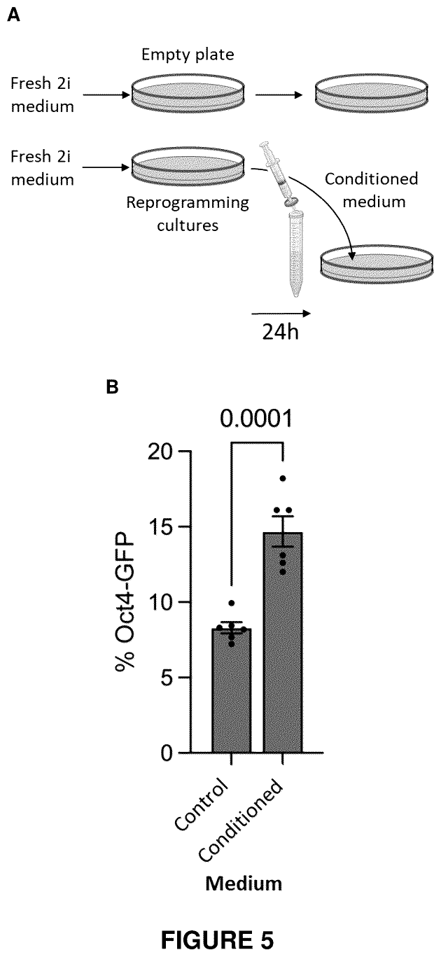

Medium was conditioned by culture on NSC reprogramming cultures (FIG. 5A, “Conditioned medium”, bottom panel) or by incubated in empty plates lacking cells under the same conditions (FIG. 5A, “Control medium”, top panel) and added to new reprogramming (receiving) cultures after every 24 h. Reprogramming efficiency of the receiving cultures was assessed by flow cytometry based GFP quantification at the end of the reprogramming assay (day 15).

As shown in FIG. 5B, the addition of medium conditioned by reprogramming cultures promoted/enhanced the proportion of OCT4-GFP positive cells, i.e. the proportion of reprogramming cells, in culture. Therefore, a signal or signalling agent secreted during reprogramming can be used to promote/enhance reprogramming, such as in distinct reprogramming cultures.

### Example 5: Citrullinated Chromatin Is Extracellular and H3Cit Can Be Isolated From Conditioned Medium from Reprogramming Cultures

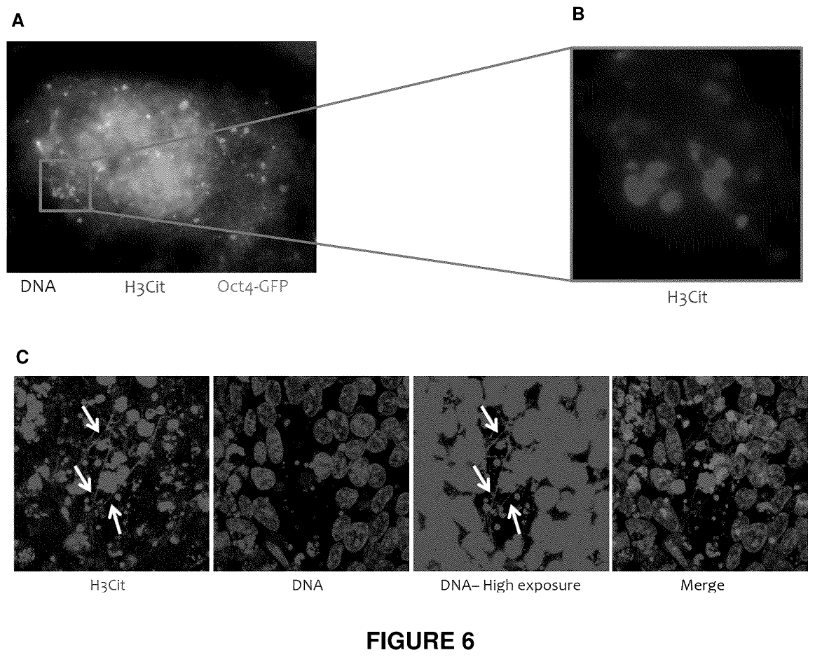

Using immunofluorescence optimised for the visualisation of extracellular, NET-like chromatin, reprogramming cultures were further analysed for H3Cit staining (FIG. 6). As shown in FIG. 6C, H3Cit positive cells undergo a process similar to NETosis, the release of chromatin, in particular citrullinated chromatin, into the extracellular space.

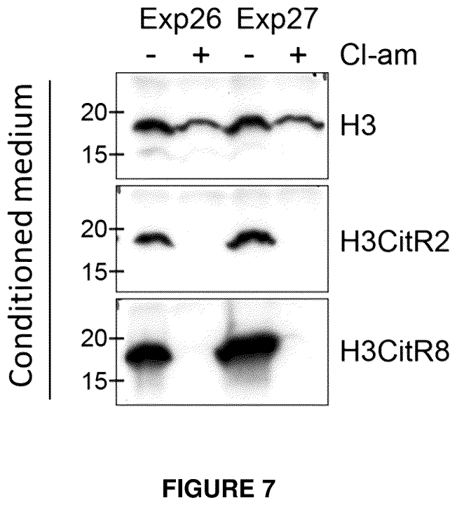

Based on this finding, the conditioned medium of Example 4 was analysed for the presence of H3Cit by Western blot (FIG. 7). Citrullinated histone H3 (H3Cit) can be readily detected in the conditioned medium from reprogramming cultures (FIG. 7, middle and lower panels labelled “H3CitR2” and H3CitR8”, lanes labelled “-CI-am”), and this is reduced in conditioned medium from reprogramming cultures in which the PADI4 inhibitor CI-amidine has been added (FIG. 7, middle and lower panels labelled “H3CitR2” and H3CitR8”, lanes labelled “+CI-am”), despite similar levels of total histone H3 being present (FIG. 7, top panel labelled “H3”).

Therefore, these data demonstrate that reprogramming cultures release citrullinated histone H3 into the culture medium in a PADI4-dependent manner.

### Example 6: NET-Like Citrullinated Chromatin is Induced During In Vivo Reprogramming and Associates With Tissue Reprogramming

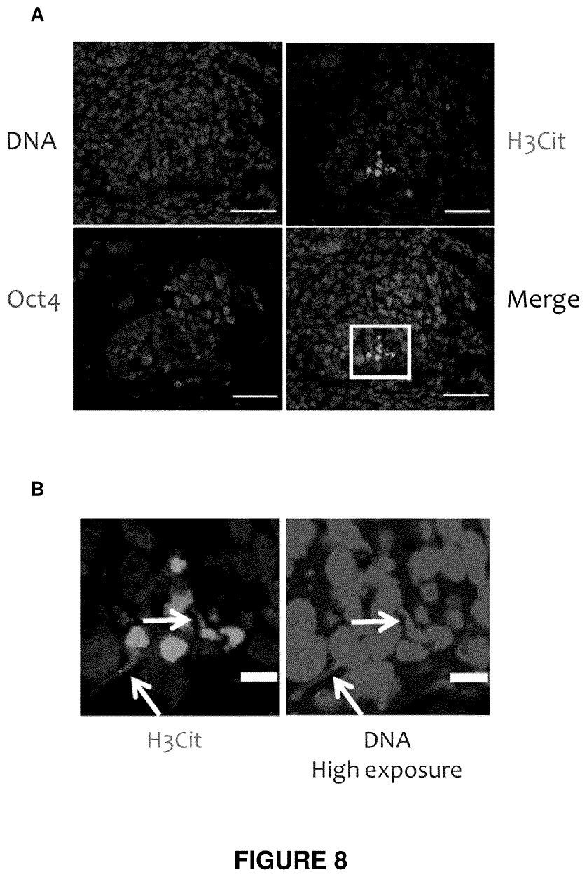

To observe the release of H3Cit in vivo during reprogramming, the mouse model described in Abad et al. (2013) Nature, 502:340-345 (doi: https://doi.org/10.1038/nature12586) was used. This mouse model expresses the Yamanaka factors in vivo and reprogramming is observed as local tissue dysplasia (de-differentiation). NET (neutrophil extracellular trap)-like structures can be seen in the regions of tissue dysplasia (i.e. the regions of reprogramming) in the stomach (FIG. 8), pancreas and colon (data not shown) of these mice. Furthermore, the amount of H3Cit staining seen in vivo correlates with the amount of reprogramming in these tissues, and none is observed in tissue where Yamanaka factors are not activated (data not shown).

Therefore, these data demonstrate the presence of extracellular citrullinated histone H3 in an in vivo model of reprogramming, specifically in those tissues undergoing reprogramming.

### Example 7: Histone Citrullination and Net-Like Chromatin Release Are Induced During the Regenerative Phase in a Model of Mouse Digit Tip Amputation and Regeneration

To further confirm the results in Example 6 seen in vivo, and assess their relevance in tissue regeneration, a mouse model of tissue regeneration was used, where the digit tip is amputated and the regeneration of the tissue is observed.

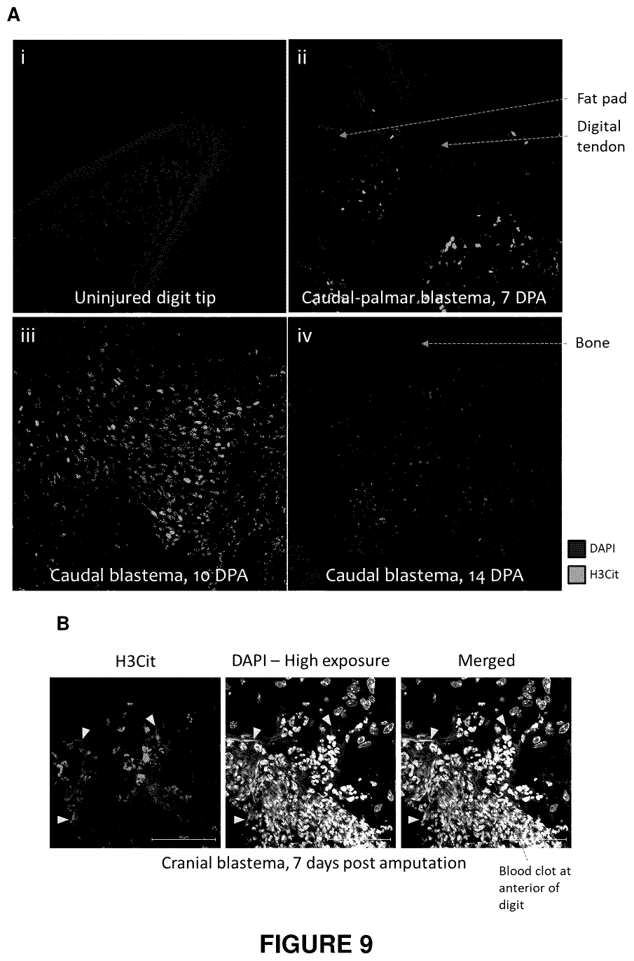

As shown in FIG. 9A, H3Cit staining can be observed in the regenerating tissue 7 days after amputation, with the peak at 10 days post amputation. FIG. 9B shows the H3Cit in higher magnification at 7 days post amputation, under which extracellular citrullinated and decondensed chromatin in NET-like structures can be seen. As such, these data confirm that seen in Example 6 and further demonstrate the presence of extracellular citrullinated histone H3 in an in vivo model of reprogramming, specifically in those tissues undergoing regeneration/rejuvenation.

### Example 8: Inhibition of Extracellular Chromatin Component Sensing Pathways Inhibits Reprogramming

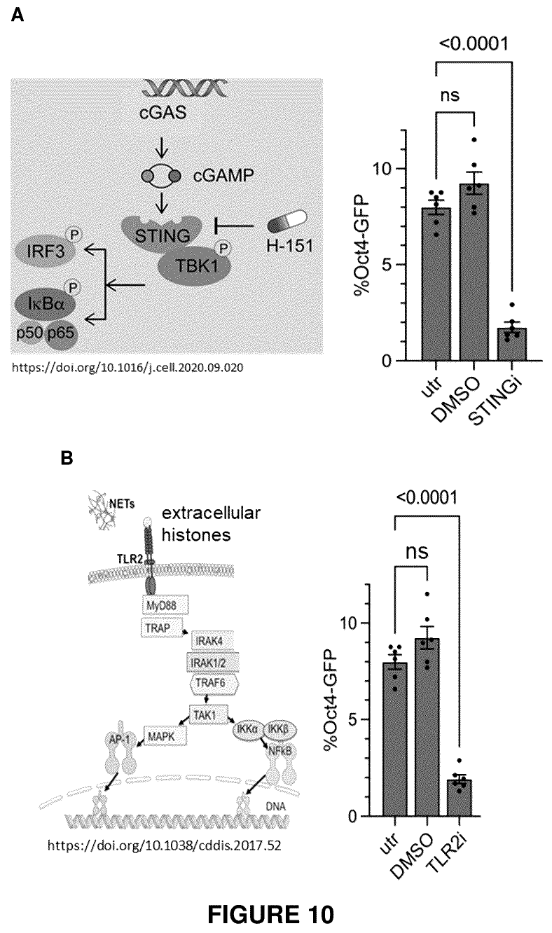

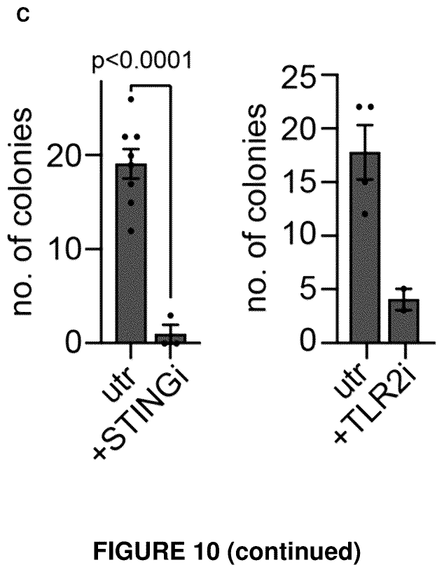

To investigate the mechanisms by which the reprogramming cells sense or respond to the extracellular chromatin/citrullinated histone H3, the STING inhibitor H-151 (STINGi) and TLR2 inhibitor MMG-11 (TLR2i) were added to neural stem cell reprogramming cultures. As shown in FIGS. 10A and 10B, the proportion of OCT4-GFP positive cells (i.e. the proportion of reprogramming cells) is significantly reduced when either the cGAS/STING or TLR pathways are inhibited. The same results are seen with a significantly reduced number of colonies in the alternative reprogramming model using human fibroblasts when the cGAS/STING or TLR pathways are inhibited (FIG. 10C).

### Example 9: Degradation of Extracellular DNA in the Medium of Reprogramming Cultures Reduces Reprogramming

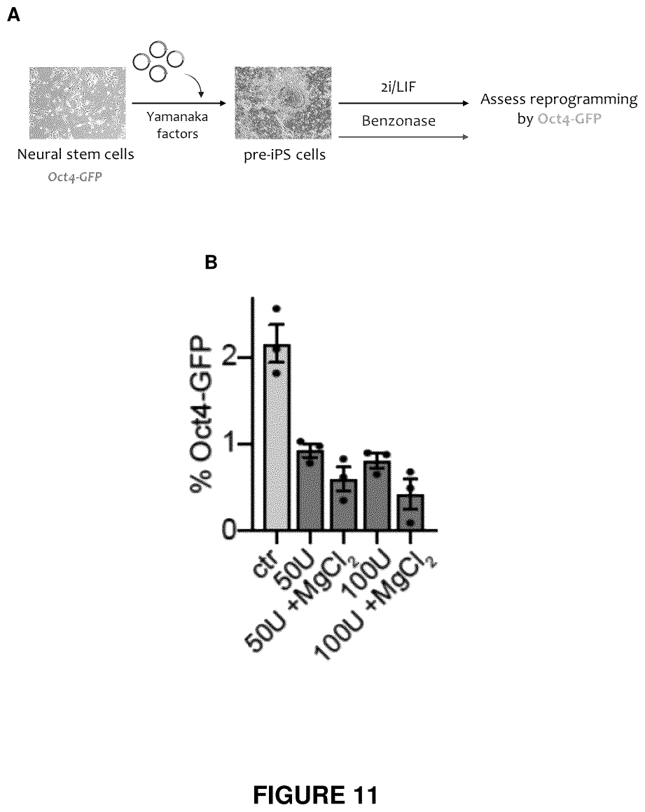

To further investigate whether extracellular chromatin/H3Cit/DNA can enhance/promote reprogramming, reprogramming cultures were treated daily with the DNA nuclease benzonase, from the pre-iPS stage until the end of the reprogramming experiment (FIG. 11A).

As can be seen in FIG. 11B, the degradation of extracellular DNA in the medium of reprogramming cultures reduces the proportion of OCT4-GFP positive cells. Thus, similar to in Example 8 where the extracellular chromatin/DNA pathways were inhibited, this data further demonstrates that extracellular chromatin/DNA may be the non-cell autonomous reprogramming factor defined herein.

### Example 10: Blocking Histones in the Medium of Reprogramming Cultures Reduces Reprogramming

To support the data in Example 5 that extracellular histones can be detected in conditioned medium and the data in Example 9 that degradation of extracellular DNA reduced reprogramming, extracellular histones were blocked/removed using small polyanions MTS which are highly charged anionic compounds that neutralise the positively charged histones (O'Meara et al. (2020) Nat. Comms., 11(6408), doi: https://doi.org/10.1038/s41467-020-20231-y).

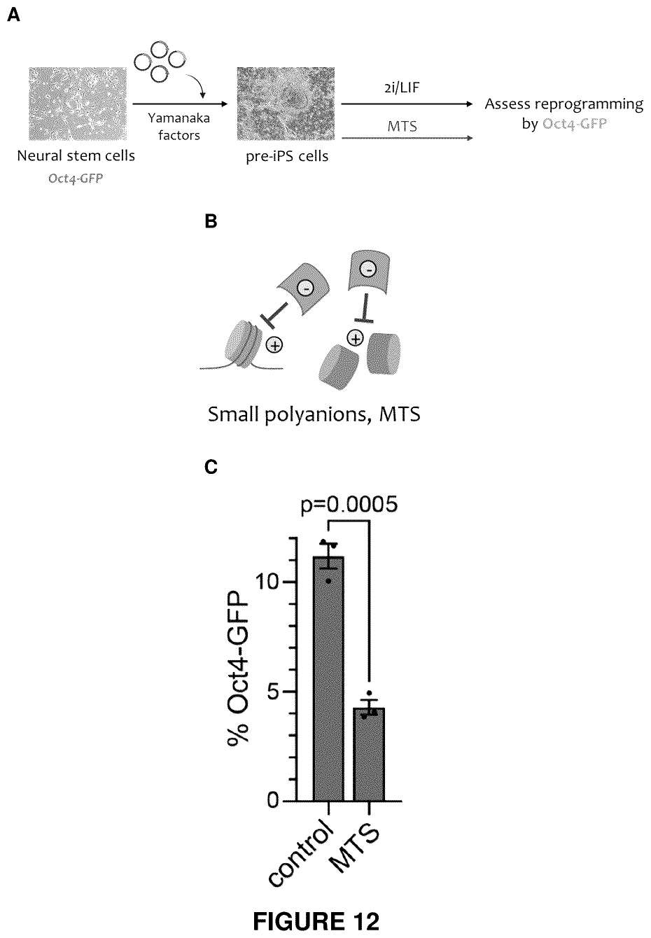

As can be seen in FIG. 12, treatment with MTS during the window of PADI4 activation led to a 60% reduction in reprogramming efficiency. Thus, similar to in Example 9 where the degradation of extracellular DNA affected reprogramming, this data further suggests that extracellular histones are involved in mediating reprogramming, in particular that histones within extracellular chromatin/DNA complexes may be the non-cell autonomous reprogramming factor defined herein.

### Example 11: The Transcription Factor c-Myc is Sufficient to Induce Expression and Activation of PADI4 and Release of Extracellular Citrullinated Chromatin

c-Myc is a potent oncogene and it would thus be desirable to omit from reprogramming cultures. To further understand the mechanisms behind release of non-cell autonomous reprogramming factors from non-reprogramming cells and to determine if c-Myc could be omitted as a cell autonomous reprogramming factor from cultures, the affect of c-Myc on PADI4 activity and citrullinated histone H3 levels was investigated.

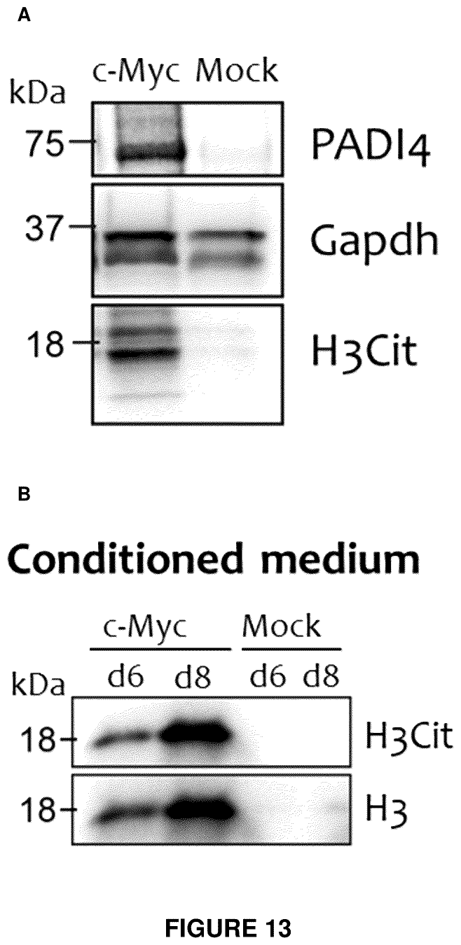

As shown in FIG. 13, c-Myc expression in neural stem cells is sufficient to induce expression and activity of PADI4 during reprogramming, with activity being demonstrated by the presence of citrullinated histone H3 in transduced cells (FIG. 13A). Furthermore, c-Myc expression is sufficient to induce the release of extracellular citrullinated histones in these cultures (FIG. 13B). Thus, it is hypothesised that the effects of c-Myc could be recapitulated using a non-cell autonomous reprogramming factor since c-Myc promotes the release of said factors into the extracellular culture medium.

### Example 12: Extracellular Citrullinated Histones Interact With Cell Surface Receptor Toll-Like Receptor 2 (TLR2)

To yet further support the data in Example 5 that extracellular histones can be detected in conditioned medium, the data in Example 8 that reprogramming is reduced in culture upon cGAS/STING or TLR pathway inhibition and the data in Example 10 that blocking histones leads to a reduction of reprogramming in culture, Toll-like receptor 2 (TLR2) was immunoprecipitated and the co-immunoprecipitated interacting proteins analysed.

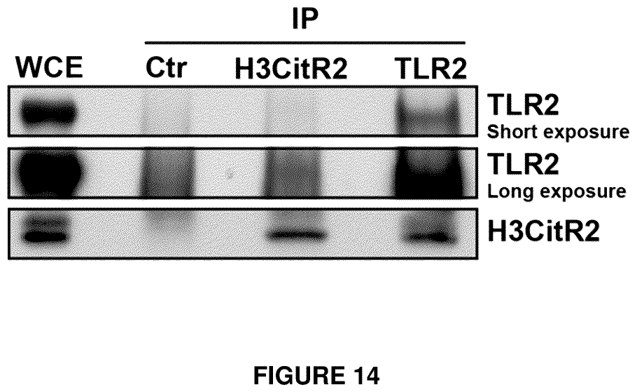

As can be seen in FIG. 14, immunoprecipitation of TLR2 results in co-precipitation of exogenous citrullinated histone H3. Thus, this data directly supports the results seen in Examples 8 and 10 that suggest exogenous histones may be the non-cell autonomous reprogramming factor as defined herein and that they are sensed by at least the TLR pathway in reprogramming cells. As such, a model is hypothesised that extracellular citrullinated histones enhance reprogramming via TLR2 signalling.

### Example 13: Extracellular Citrullinated Histones are Induced During In Vivo Tissue Regeneration After DSS-Induced Colitis

The data in Examples 6 and 7, that extracellular citrullinated histone H3 is observed in in vivo models of reprogramming, was further confirmed in a model of chemical insult-induced injury (Dextran Sulfate Sodium (DSS)-induced colitis).

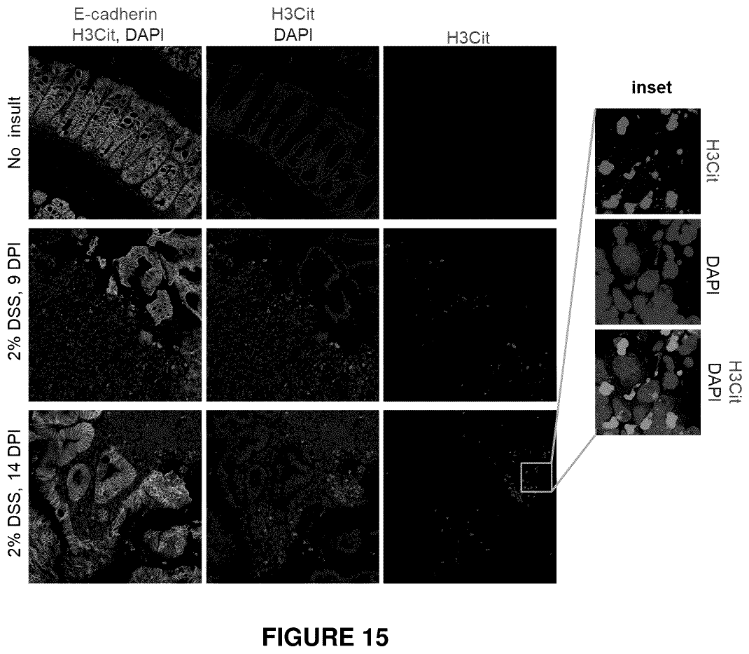

As can be seen in FIG. 15, H3Cit is also induced in regeneration after DSS insult-induced injury, and supports the findings hereinbefore that it is induced in in vivo reprogramming (Example 6) and in a model of physical insult (digit tip regeneration; Example 7). It also shows that, like in the other models, H3Cit is associated with NET-like structures (FIG. 15, inset).

### Example 14: the Marker of Repairing Epithelium, Ly6a, Is Expressed Together With PADI4 in Non-Reprogramming Cells

Ly6a (also known as Sca-1) is a marker that has been shown to associate with an “alternative” (i.e. non-iPS) cell fate in reprogramming in vitro (Schwarz et al. (2018) Cell Stem Cell, doi: https://doi.org/10.1016/j.stem.2018.06.013) and in vivo (Chondronasiou et al. (2022) Stem Cell Reports, doi: https://doi.org/10.1016/j.stemcr.2022.09.009). Importantly, Ly6a/Sca-1 has also been shown to mark the repairing epithelium in a model of tissue regeneration after Dextran Sulfate Sodium (DSS)-induced colitis (Yui et al. (2018) Cell Stem Cell, doi: https://doi.org/10.1016/j.stem.2017.11.001).

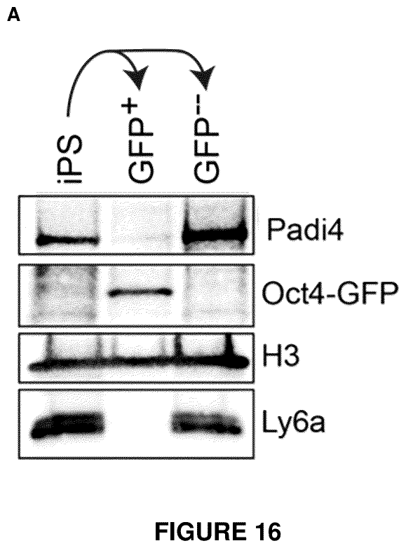

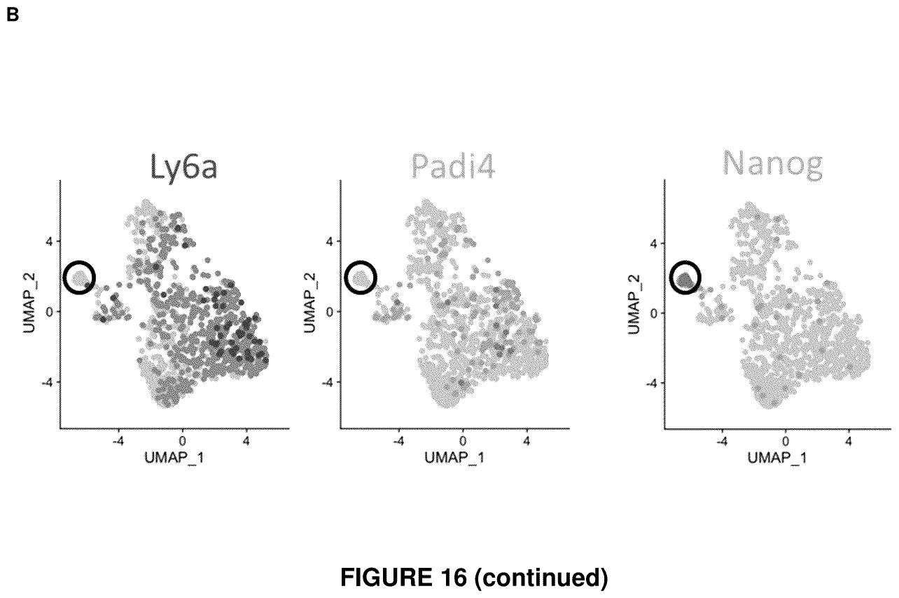

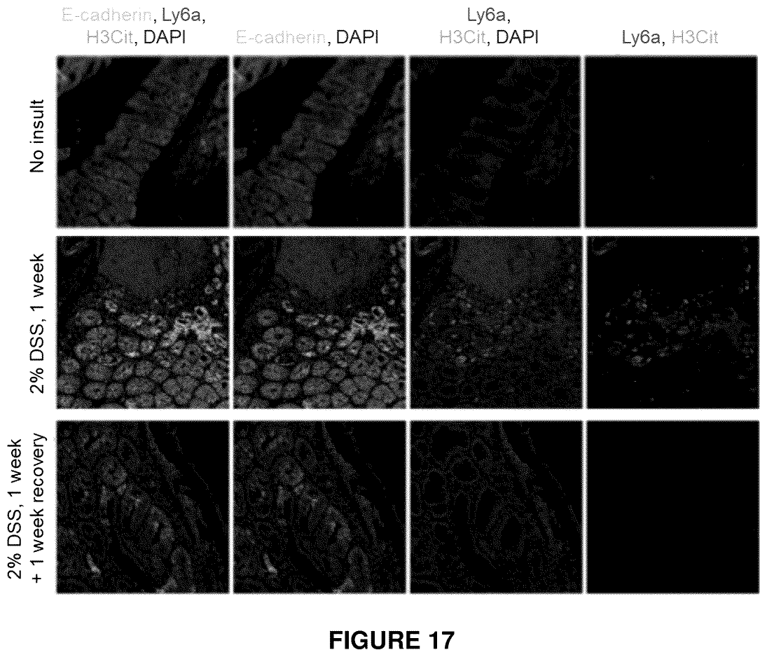

As shown in FIG. 16, Ly6a is expressed in GFP-negative and Nanog-negative, non-reprogrammed cells, similarly to PADI4. Given that Ly6a has been identified as a marker of the repairing epithelium (Yui et al. (2018)) and it is found herein to be associated in the same cell population as PADI4 (i.e. non-reprogramming cells), this further supports the hypothesis that PADI4 is a marker of repair/regeneration. This is yet further supported by Example 15 and the data shown in FIG. 17.

### Example 15: Ly6a and H3Cit Are Induced During the Regeneration Phase and Their Expression is Localised in the Repairing Epithelium

To support the data in Example 7 that extracellular citrullinated histone H3 can be seen in an in vivo model of reprogramming and in Example 14 that PADI4 expression associates with Ly6a expression, the expression of these markers of regeneration were analysed at different phases of tissue regeneration after DSS-induced injury.

As shown in FIG. 17, Ly6a and H3Cit are induced during the regeneration phase (FIG. 17, middle panel) and their expression is localised in the repairing epithelium (as shown previously for Ly6a by Yui et al. (2018)). This still further supports the data hereinbefore in Example 7 (FIG. 9), that PADI4 activation (as evidenced by the presence of extracellular H3Cit) is associated with the regeneration phase, and also data presented in Example 15 (FIG. 16) that PADI4 expression associates with Ly6a expression.

Taken together, these data presented herein demonstrate that the release of a non-cell autonomous reprogramming factor, such as extracellular chromatin, DNA and/or citrullinated histone H3, by non-reprogramming cells can promote or enhance the reprogramming of reprogramming cells either within the same culture or in distinct reprogramming cultures, such as via conditioned medium exchange. Said non-cell autonomous reprogramming factor may also supplement or even replace one or more cell-autonomous reprogramming factor which are otherwise required to drive reprogramming (i.e. one or more Yamanaka factors). The non-cell autonomous reprogramming factor released by the non-reprogramming cells is sensed by the reprogramming cells, and the inhibition of extracellular chromatin/DNA sensing pathways, such as the cGAS/STING or TLR pathways, the degradation of extracellular DNA, or the blocking/neutralisation of extracellular histones reduces reprogramming, indicates that said non-cell autonomous reprogramming factor may be extracellular chromatin, DNA and/or citrullinated histone H3. The finding herein that citrullinated histone H3 co-immunoprecipitates with TLR2 further supports this. The activation and/or expression of proteins involved in the release/secretion of the non-cell autonomous reprogramming factor, in particular citrullinated histone H3, such as PADI4 may be used as markers of regeneration in vivo, and the data herein showing the co-expression of PADI4 with the known marker of repairing epithelium, Ly6a, demonstrates an active role in tissue regeneration for pathways involved in chromatin, DNA and/or histone release/secretion.

1. A method of non-cell autonomous modulation of reprogramming, comprising providing a non-cell autonomous reprogramming factor and a somatic cell to be reprogrammed, wherein said non-cell autonomous reprogramming factor derives from a cell other than the somatic cell to be reprogrammed.

2. The method of claim 1, wherein the cell other than the somatic cell to be reprogrammed is a non-reprogramming cell, such as a bystander cell.

3. The method of claim 1, wherein the non-cell autonomous reprogramming factor derives from a non-reprogramming cell, such as a bystander cell, and/or
wherein the non-cell autonomous reprogramming factor does not derive from the somatic cell to be reprogrammed, such as does not derive from the reprogramming cell.

4. The method of claim 1, wherein the non-cell autonomous reprogramming factor is released from the cell other than the somatic cell to be reprogrammed, such as is released from a non-reprogramming cell and/or a bystander cell, and/or
wherein the non-cell autonomous reprogramming factor is secreted from the cell other than the somatic cell to be reprogrammed, such as is secreted from a non-reprogramming cell and/or a bystander cell.

5. The method of claim 4, wherein the method is performed in an in vitro reprogramming culture and the non-cell autonomous reprogramming factor is released or secreted into the culture medium.

6. The method of claim 1, wherein the non-cell autonomous reprogramming factor is chromatin or a component thereof, or a functional analogue thereof.

7. The method of claim 6, wherein the non-cell autonomous reprogramming factor is an extracellular nucleosome or a component thereof, or a functional analogue thereof, and/or
wherein the non-cell autonomous reprogramming factor is extracellular DNA or a functional analogue thereof, and/or
wherein the chromatin comprises histone H3 or a functional analogue thereof, such as wherein the non-cell autonomous reprogramming factor is histone H3.

8. The method of claim 6, wherein the chromatin or component thereof is modified, such as post-translationally modified.

9. The method of claim 8, wherein the chromatin or component thereof is citrullinated, and/or
wherein the chromatin or component thereof comprises a citrullinated histone.

10. The method of claim 9, wherein the citrullinated histone is citrullinated histone H3.

11. The method of claim 1, wherein the non-cell autonomous reprogramming factor is an agonist of a chromatin sensing pathway.

12. The method of claim 1, wherein the method additionally comprises providing a non-reprogramming cell and/or a bystander cell.

13. The method of claim 1, wherein the method is performed in vivo, in vitro or ex vivo, such as in an in vitro reprogramming culture.

14. The method of claim 1, wherein one or more cell autonomous reprogramming factors are provided which derive from the somatic cell to be reprogrammed, such as the reprogramming cell.

15. The method of claim 14, wherein the one or more cell autonomous reprogramming factors are Yamanaka factors, such as a Yamanaka factor selected from one or more of: OCT4, KLF4, c-MYC, SOX2, LIN28, NANOG, ESSRRB, NR5A2 and/or C/EBPα, in particular one or more of: OCT4, KLF4, c-MYC and SOX2, and/or
wherein the one or more cell autonomous reprogramming factors are expressed by the somatic cell to be reprogrammed, such as wherein the one or more cell autonomous reprogramming factors are expressed from a nucleic acid sequence encoding said cell autonomous factors in the somatic cell to be reprogrammed.

16. The method of claim 14, wherein the non-cell autonomous reprogramming factor enhances or promotes the reprogramming effect of the one or more cell autonomous reprogramming factors.

17. The method of claim 1, wherein reprogramming is full reprogramming or is partial and/or incomplete reprogramming.

18. (canceled)

19. (canceled)

20. (canceled)

21. An in vivo method of non-cell autonomous modulation of reprogramming, said method comprising administering the non-cell autonomous reprogramming factor defined in claim 3 to a subject.

22. A method of treating and/or ameliorating a degenerative disease or disorder in a subject, said method comprising administering to the subject the non-cell autonomous reprogramming factor defined in claim 3 or comprising the in vivo method of non-cell autonomous modulation of reprogramming of claim 21.
